(19) United States
(12) Patent Application Publication
(10) Pub. No.: US 2023/0390192 A1
(43) Pub. Date: Dec. 7, 2023

(54) GASTRO RETENTIVE DOSAGE FORMS COMPRISING DEUTETRABENAZINE

(71) Applicant: AUSPEX PHARMACEUTICALS, INC., Parsippany, NJ (US)

(72) Inventors: Mayank JOSHI, Parsippany, NJ (US); Parag SHAH, Parsippany, NJ (US); Soumen PATTANAYEK, Parsippany, NJ (US); Divyang PATEL, Parsippany, NJ (US); Sandeep PANDITA, Parsippany, NJ (US)

(21) Appl. No.: 18/246,582

(22) PCT Filed: Oct. 12, 2021
(86) PCT No.: PCT/US2021/054511

§ 371 Date: Mar. 24, 2023

Related U.S. Application Data
(60) Provisional application No. 63/090,546, filed on Oct. 12, 2020.

(51) Int. Cl. A61K 9/00; A61K 31/4745; A61K 9/20; A61K 47/32; A61K 47/38

(52) U.S. Cl. CPC A61K 9/0065; A61K 31/4745; A61K 9/2077; A61K 47/32; A61K 47/38

(57) ABSTRACT

Provided herein are controlled release gastro retentive dosage forms containing deutetrabenazine for use in the treatment of, e.g., hyperkinetic movement disorders. When orally administered to a subject on a once-daily basis, the dosage forms provide a favorable pharmacokinetic profile.

### CROSS REFERENCE TO RELATED APPLICATIONS

This application claims the benefit of U.S. Provisional Application No. 63/090,546, filed Oct. 12, 2020, the entirety of which is incorporated herein by reference.

### TECHNICAL FIELD

The present disclosure pertains to gastro retentive dosage forms, manufacturing processes and methods of use of the gastro retentive dosage forms for treating hyperkinetic movement disorders deriving from conditions including Huntington's disease, tardive dyskinesia, levodopa-induced dyskinesia and dyskinesia in cerebral palsy.

### BACKGROUND

Deutetrabenazine ((RR,SS)-1,3,4,6,7,11b-hexahydro-9,10-di(methoxy-D3)-3-(2-methylpropyl)-2H-benzo[a]quinolizin-2-one) is a vesicular monoamine transporter type 2 (VMAT2). The biologically active metabolites formed from deutetrabenazine (alpha-dihydrodeutetrabenazine [α-deuHTBZ] and beta-dihydrodeutetrabenazine [β-deuHTBZ]), together identified as “deuHTBZ”, are potent inhibitors of VMAT2 binding. Deutetrabenazine exhibits an increased half-life of its active metabolites, relative to tetrabenazine (e.g., U.S. Pat. No. 8,524,733).

Deutetrabenazine (deu-TBZ) is approved by the U.S. Food and Drug Administration under the tradename AUSTEDO® for the treatment of chorea (involuntary muscle movements) associated with Huntington's disease (HD) and for the treatment of tardive dyskinesia (TD) in adults. AUSTEDO® dosage forms are orally administered twice-daily (bid), for total daily dosages of 12 mg or above of deutetrabenazine.

Several factors affect gastrointestinal absorption of orally administered drugs including solubility of the drug at various pH and the rate at which drug is released from the dosage form. Drug release rates for oral dosage forms are typically measured as rate of dissolution in vitro, i.e., a quantity of drug released from the dosage form per unit time in, for example, an FDA approved system. Such systems include, for example, United States Pharmacopeia (USP) dissolution apparati I, II and III.

The therapeutic window of a drug is the period when the plasma drug concentration is within the therapeutically effective plasma drug concentration range. Because the plasma drug concentration declines over time, however, multiple doses of drug dosage form must be administered at appropriate intervals to ensure that the plasma drug concentration remains within or, again rises to, the therapeutic window. At the same time, however, there is a need to avoid or minimize plasma drug concentrations that result in undesirable side effects.

Several dosage forms comprising deutetrabenazine are disclosed in U.S. Pat. No. 9,296,739. A dosage form that can deliver deutetrabenazine in a controlled manner over an extended period would enable amore advantageous dosing regimen, e.g., one that would permit once-daily (“qd”) administration while maintaining the treatment effects currently realized by AUSTEDO®. A need exists for such alternative dosage forms.

### SUMMARY

Disclosed herein are controlled release gastro retentive dosage forms for once daily oral administration of deutetrabenazine to a subject in need thereof.

The controlled release oral dosage forms of the invention comprise an amount of deutetrabenazine, at least two control release polymers, each independently having a viscosity of 2,000 cPs or more, and a pharmaceutically acceptable excipient comprising at least one disintegrant, wherein the total amount of control release polymers being at least 30 wt. % relative to the total weight of the dosage form.

In other embodiments, the dosage forms include a pharmaceutically acceptable excipient comprising at least one disintegrant and at least one of a diluent, a binder, a gas-generating agent, an antioxidant, a lubricant or a combination thereof.

In some embodiments, the controlled release gastro retentive dosage forms comprise deutetrabenazine having a median particle size of 1 to 30 micron (μm).

In yet other embodiments, the invention provides methods useful in treating VMAT2 mediated disorders. In some embodiments, the method of treating a VMAT2 mediated disorder comprises orally administering to a patient in a need thereof, the controlled release dosage form disclosed herein.

### BRIEF DESCRIPTION OF THE DRAWINGS

FIG. 1 shows the dissolution profiles of Samples 1, 2, and 3 in 500 mL acid buffer pH 3, USPII apparatus, 75 rpm with a sinker. The co-milled and micro-milled particles exhibit a slower dissolution profile in pH 3 than an un-milled sample. The triangles represent release profile of the micro-milled sample, showing slow release (˜50% after 14 hr); the squares represent the release profile of the co-milled deutetrabenazine and the diamonds represent the release profile of the unmilled deutetrabenazine.

FIG. 2 is a graph showing the dissolution profiles for Samples 4-7 in pH 3.0 phthalate buffer, USPII. 75 rpm with a sinker.

FIG. 3 is a graph showing the dissolution profiles for Samples 8-9 in pH 3.0 phthalate buffer, USPII. 75 rpm with a sinker.

FIG. 4 is a graph showing the dissolution profiles for Samples 10-11 in pH 3.0 phthalate buffer, USPII, 75 rpm with a sinker.

FIG. 5 is a graph showing the dissolution profiles for Samples 12-13 in pH 3.0 phthalate buffer, USPII, 75 rpm with a sinker.

FIG. 6 is a graph showing the dissolution profiles for Samples 14-17 in pH 3.0 phthalate buffer, USPII, 75 rpm with a sinker.

FIG. 7 is a graph showing the dissolution profiles for Samples 18-19 in pH 3.0 phthalate buffer, USPII, 75 rpm, with a sinker.

FIG. 8 is a graph showing the dissolution profiles for Samples 20-21 in pH 3.0 phthalate buffer, USPII, 75 rpm with a sinker.

FIG. 9 is a graph showing the dissolution profiles for Samples 22-23 in pH 3.0 phthalate buffer, USPII, 75 rpm, with a sinker.

### DETAILED DESCRIPTION OF THE INVENTION

The present subject matter may be understood more readily by reference to the following detailed description, which forms a part of this disclosure. It is to be understood that this invention is not limited to the specific methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art.

As employed above and throughout the disclosure, the following terms and abbreviations, unless otherwise indicated, shall be understood to have the following meanings.

The singular forms “a,” “an,” and “the” may refer to plural articles unless specifically stated otherwise.

The term “about,” as used herein, is intended to qualify the numerical values which it modifies, denoting such a value as variable within a margin of ±10%.

When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

As used herein, the terms “compound,” “drug,” “pharmacologically active agent,” “active agent,” or “medicament” are used interchangeably herein to refer to a compound or compounds or composition of matter which, when administered to a subject (human or animal) induces a desired pharmacological and/or physiologic effect by local and/or systemic action.

The active agent disclosed herein is preferably deutetrabenazine. “Deutetrabenazine” is a selectively deuterium-substituted, stable, non-radioactive isotopic form of tetrabenazine in which the six hydrogen atoms on the two O-linked methyl groups have been replaced with deuterium atoms (i.e. —OCD₃ rather than —OCH₃ moieties).

As used herein, “dosage form” refers to an oral drug form having gastro retentive properties, as described herein.

“Controlled release” refers to a dosage form able to release active agent over an extended period for example, up to about 12 hours, 15 hours, 18 hours, 21 hours or up to about 24 hours. The active agent is preferably deutetrabenazine, as disclosed herein. In some embodiments, the dosage form releases about 20 wt. %-40 wt. % of the active agent in the dosage form within two hours and about 60 wt. %-80 wt. % within 8 hours as measured in a USPII apparatus, pH 3. In other embodiments, the dosage form releases about 40-60 wt % of the active agent in the dosage form within 7 hours, as measured in a USPII apparatus, pH 3. In other embodiments, the dosage form releases about 50 wt % of deutetrabenazine within 7 hours, as measured in a USPII apparatus, pH 3.

As used herein, the viscosity of a polymer means the viscosity measured at 2 wt. % polymer in aqueous solution at about 20-25° C.

The controlled release solid oral dosage forms of the invention remain in the gastric region (i.e. stomach) for several hours, for example up to 24 hours (prevented from moving further through the gastrointestinal tract) and hence significantly prolong the gastric residence time of the active agent. Preferably, controlled release solid oral dosage forms of the invention remain in the stomach for 6-20 hours. Prolonged gastric retention improves bioavailability, reduces drug waste and may improve availability for drugs that are less soluble in a high pH environment, i.e. in the intestinal milieu. Without being bound to any mechanism or theory, the extended gastric retention can be at least partially based on gas generation by one or more excipients within the dosage form and/or the mucoadhesive properties of the formulation. To achieve mucoadhesive properties, a number of bioadhesive polymers can be used. Such polymers swell in water and attach to mucosal surfaces. Swelling of specific polymers, optionally in combination with additional excipients (i.e., disintegrants) within the dosage form provide floating properties upon contact with the gastric milieu. The controlled release solid oral dosage forms described herein preferably swell to a size that is at least about twice its unswelled volume, upon contact with gastric fluids.

As used herein, “floating” is used in conjunction with a “floating gastro retentive dosage form” which exhibits a bulk density lower than that of the gastric fluids [Timmermans, J. and Moes, A. J., (1990) How well do floating dosage forms float? Int. J Pharm. 62: 207-216]. Such dosage forms are “floating” in that they remain buoyant in the gastric fluids of the stomach for a targeted period. The floating dosage form is then able to be retained in the stomach, while releasing an active agent.

The terms “swellable” and “swelling,” as used herein with respect to a polymer within the dosage forms, refer to a polymer capable of imbibing fluid and enlarging in size, i.e. swelling when coming in contact with a fluid environment, such as the gastric milieu.

As used herein, the terms “method of treatment” or “therapy” (as well as different forms thereof) include preventative (e.g., prophylactic), curative, or palliative treatment. As used herein, the term “treating” includes alleviating or reducing at least one adverse or negative effect or symptom of a condition, disease or disorder. This condition, disease or disorder may refer to hyperkinetic movement disorder, such as, but not limited to, chorea associated with Huntington's disease, tardive dyskinesia, Tourette syndrome, dystonia, dyskinesia in cerebral palsy (DCP) and levodopa-induced dyskinesia (LID) in Parkinson's disease.

The term “administering” means providing to a patient the pharmaceutical composition or dosage form (used interchangeably herein) of the present invention.

The terms “subject,” “individual,” and “patient” are used interchangeably herein, and refer to a human, to whom treatment, including prophylactic treatment, with a dosage form described herein, is provided.

“Pharmaceutically acceptable” refers to those compounds, materials, compositions, and/or excipients which are, within the scope of sound medical judgment, suitable for contact with the tissues of human beings without excessive toxicity, irritation, allergic response, or other problem complications commensurate with a reasonable benefit/risk ratio.

As used herein, the term “particle size distribution” refers to the statistical distribution of the volume share related of all particle sizes and is represented by D90, D50 or D10 values. The D90 value of the integral volume distribution is defined in the context of this invention as the particle diameter, at which 90 percent by volume of the particles have a smaller diameter than the diameter, which corresponds to the D90 value. For example, a D90 of 15 μm, means that 90% (by volume) of the particles have a diameter less than or equal to 15 μm. A D50 of 10 μm, means that 50% (by volume) of the particles have a diameter less than or equal to 10 μm. A D10 of 3 μm, means that 10% (by volume) of the particles have a diameter less than or equal to 3 μm. Particle size distribution is determined by means of laser diffractometry. More specifically, the particle size distribution is determined using a Mastersizer 3000 from Malvern Instruments.

As used herein, “control release” and release controlling” are used interchangeably and refer to the characteristic of the polymers used in the dosage form. Such polymers when included in a dosage form with an API are able to slow the release of the API under physiological conditions, compared to an immediate release formulation, and contribute to the release of the active agent over an extended period.

Although constant-release dosage forms have been proven effective for many different drug therapies, there are clinical situations where these have not been entirely satisfactory. It has been observed that for some patients, the therapeutic effectiveness of the drug decreases below the therapeutically effective threshold before the end of the desired therapy period despite the maintenance of substantially constant drug release that would be expected to provide continued effectiveness.

It has been surprisingly discovered that oral dosage forms comprising deutetrabenazine that exhibit a desirable rate of release and hence a desirable pharmacokinetic profile for an extended time can be achieved. In some embodiments, the presently disclosed gastro retentive dosage forms when administered orally to a subject on a once daily basis (qd) provide a pharmacokinetic profile that is comparable, e.g., bioequivalent, to that of the AUSTEDO® dosage forms administered twice daily (bid).

Provided herein are controlled release gastro retentive oral dosage forms for once daily administration of deutetrabenazine comprising: an amount of deutetrabenazine, at least two control release polymers, each independently having a viscosity of 2,000 cPs or more, and a pharmaceutically acceptable excipient comprising at least one disintegrant, wherein the total amount of control release polymers being at least 30 wt. % relative to the total weight of the dosage form.

The dosage forms of the disclosure include a total of 2-15 wt. % deutetrabenazine, relative to the total weight of the dosage form. In some embodiments, the dosage forms include a total of 3-10 wt. % deutetrabenazine, relative to the total weight of the dosage form. In some embodiments, the dosage forms include a total of 4-7 wt. % deutetrabenazine, relative to the total weight of the dosage form. In certain embodiments, the dosage forms include a total of 2, 3, 4, 5, 6, 7, 8, 9, 10, 11, 12, 13, 14, 15 wt. % deutetrabenazine, relative to the total weight of the dosage form.

The dosage forms further include two or more control release polymers. In some embodiments, each of the two control release polymers independently has a viscosity within the range of 2,000 to 80,000 cPs. In some embodiments, each of the two control release polymers independently has a viscosity of 2,000 to 60,000 cPs, 2,000 to 40,000 cPs, 2,000 to 20,000 cPs, 2,000 to 10,000 cPs, 2,000 to 8,000 cPs, 2,000 to 6,000 cPs or 2,000 to 4,000 cPs. In one specific embodiment, each of the two control release polymers independently has a viscosity of 2,000 to 4,000 cPs. In some embodiments, each of the two control release polymers independently has a viscosity of 10,000 to 80,000 cPs, 20,000 to 80,000 cPs 30,000 to 70,000 cPs or 40,000 to 60,000 cPs. In one specific embodiment, each of the two control release polymers independently has a viscosity of 40,000 to 60,000 cPs. In one specific embodiment, at least one of the control release polymers has a viscosity of 2,000 to 4,000 cPs and at least one of the control release polymers has a viscosity of 40,000 to 60,000 cPs.

The dosage forms may optionally further include one or more additional control release polymers, having a viscosity of about 3 to about 80,000 cPs. In some embodiments, the one or more additional control release polymers independently have a viscosity of 3 to 60,000 cPs, 3 to 40,000 cPs, 3 to 20,000 cPs, or 3 to 15,000 cPs. In one specific embodiment, the dosage forms comprise two control release polymers each independently having a viscosity within the range of 2,000 to 80.000 cPs and one additional control release polymer having a viscosity within the range of 3 to 15,000 cPs. In yet another specific embodiment, the dosage forms comprise at least one control release polymer having a viscosity within a range of 2,000 to 4,000 cPs and at least one control release polymer having a viscosity within the range of 40,000 to 60,000 cPs and further include at least one additional control release polymer having a viscosity within the range of 3 to 15,000 cPs.

The release controlling (also referred to herein as “control release”) polymers of the disclosure comprise a water soluble polymer, a water insoluble polymer or mixtures thereof. In some embodiments, the dosage form includes a water soluble polymer. In some embodiments, the water soluble polymer is or comprises hydroxypropyl cellulose, hydroxypropyl methylcellulose, polyvinyl pyrrolidone, polyvinyl alcohol, a polyacrylic acid polymer or mixtures thereof. In a preferred embodiment, the water soluble polymers comprise hydroxypropyl methylcellulose, a polyacrylic acid polymer or mixtures thereof. In another preferred embodiment, the polyacrylic acid polymer comprises a cross-linked polyacrylic acid polymer, such as Carbopol®. In yet another preferred embodiment, the dosage forms include a water insoluble polymer. In some embodiments, the water insoluble polymer is or comprises copovidone, croscarmellose sodium, starch, sodium starch glycolate, colloidal silica, silica, ethyl cellulose, polyvinyl acetate, or mixtures thereof. In another preferred embodiment, the water insoluble polymer comprises copovidone, or mixtures thereof. In one specific embodiment, the copovidone is Kollidon®.

In some embodiments, the control release polymers in the dosage forms include a mixture of one or more water soluble and one or more water insoluble polymers. In one embodiment, the dosage forms comprise a release controlling polymer mixture of copovidone, hydroxypropyl methylcellulose and a polyacrylic acid polymer.

The release controlling polymers are present in the dosage forms in a range of 30 to 65 wt. % relative to the total weight of the solid oral dosage form. In some embodiments, the release controlling polymers are present in the dosage forms in a range of 30 to 60 wt. % relative to the total weight of the solid oral dosage form. In some embodiments, the release controlling polymers are present in the dosage forms in a range of 30 to 55 wt. % relative to the total weight of the solid oral dosage form. In some embodiments, the release controlling polymers are present in the dosage forms in a range of 35 to 55 wt. % relative to the total weight of the solid oral dosage form. In some embodiments, the release controlling polymers are present in the dosage forms in a range of 40 to 55 wt. % relative to the total weight of the solid oral dosage form. In some embodiments, the release controlling polymers are present in the dosage forms in a range of 45 to 55 wt. % relative to the total weight of the solid oral dosage form. In some embodiments, the release controlling polymers are present in the dosage forms in a range of 40 to 50 wt. % relative to the total weight of the solid oral dosage form. In some aspects, the release controlling polymers are present in the dosage forms at 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 wt. % relative to the total weight of the solid oral dosage form.

The dosage forms further include a pharmaceutically acceptable excipient, comprising at least one disintegrant. In one embodiment, the disintegrant is or comprises croscarmellose sodium, alginic acid, microcrystalline cellulose, crospovidone, polacrilin potassium, sodium starch glycolate, low-substituted hydroxypropyl cellulose, starch, and mixtures thereof.

The pharmaceutically acceptable excipient may further comprise at least one of a diluent, a binder, a gas-generating agent, an antioxidant, a lubricant or combinations thereof. Typically, a combination of pharmaceutically acceptable excipient materials is used.

The excipients may comprise a gas-generating agent. In one embodiment, the gas-generating agent comprises a carbonate salt that may be selected from sodium bicarbonate, a mixture of sodium bicarbonate and citric acid, calcium carbonate, a mixture of calcium carbonate and citric acid and a mixture thereof. The gas-generating agent is present in the dosage forms in a range of 5 to 15 wt. %, relative to the total weight of the solid oral dosage form. In some embodiments, the excipient comprises about (by wt. %) 5.0, 6.0, 7.0, 8.0, 9.0, 10.0, 11.0, 12.0, 13.0, 14.0, or 15.0 wt. %, relative to the total weight of the dosage form. In some embodiments, the excipient comprises about (by wt. %) 5, 6, 7, 8, 9, 10, 11, 12, 13, 14, or 15 wt. %, relative to the total weight of the dosage form.

The excipients may comprise an antioxidant. In one embodiment, the antioxidant is or comprises propyl gallate, 6-ethoxy-1,2-digydro-2,2,4-trimethylquinoline (ethoxyquin), nordihydroguaiaretic acid (NDGA), butylated hydroxyanisole, butylated hydroxytoluene or any mixture thereof. In a preferred embodiment, the antioxidant comprises a mixture of butylated hydroxyanisole and butylated hydroxytoluene. The antioxidant is present in the dosage form in a range of 0.1 to 1.0 wt. %, or about 0.2-1.0 wt. %, or about 0.5-0.8 wt. % relative to the total weight of the solid oral dosage form. For example, the antioxidant is present in the dosage form at 0.1, 0.2, 0.3, 0.4, 0.5, 0.6, 0.7, 0.8, 0.9 or 1.0 wt. % relative to the total weight of the solid oral dosage form.

The excipients may comprise a binder. In some embodiments, the binder comprises a water-soluble binder, a water-insoluble binder or combinations thereof. In some embodiments, the binder comprises a water-soluble binder, which may be a cellulose-based binder including hydroxypropyl cellulose, hydroxypropyl methylcellulose, polyvinyl pyrrolidone, polyvinyl alcohol, a polyacrylic acid polymer, polyether, carbohydrate polymer (natural or synthetic) or combinations thereof. In some embodiments, the binder is a cellulose-based binder selected from the group consisting of methyl cellulose (MC), ethyl cellulose (EC), propyl cellulose (PC), hydroxymethyl cellulose (HMC), hydroxyethyl cellulose (HEC), hydroxypropyl cellulose (HPC), hydroxypropyl methyl cellulose (HPMC, hypromellose (INN), cellulose acetate and combinations thereof. In some embodiments, the binder is a polyether. Suitable polyethers include polyethylene glycol (PEG) polymers. In further embodiments, the binder comprises a water-insoluble polymer, which is or comprises crospovidone, copovidone, microcrystalline cellulose, croscarmellose sodium, starch, sodium starch glycolate, colloidal silica, silica, ethyl cellulose, lactic acid polymer, a lactic acid and glutamic acid copolymer, polyvinyl acetate or combinations thereof. In some embodiments, the binder comprises microcrystalline cellulose, gelatin, agar, natural and synthetic gums or any mixture thereof. In one specific embodiment, the binder comprises microcrystalline cellulose. In some embodiments, the binder is present in the dosage form in a range of 5 to 50 wt. %, relative to the total weight of the solid oral dosage form. In a preferred embodiment, the binder is present in the dosage form in a range of 30 to 50 wt. %, relative to the total weight of the solid oral dosage form. For example, the binder is present in the dosage form at 5, 10, 15, 20, 25, 30, 35, 40, 45, or 50 wt. % relative to the total weight of the solid oral dosage form.

In some embodiments, the excipients comprise a diluent selected from the group consisting of a saccharide, a disaccharide, a polysaccharide, a polyalcohol, microcrystalline cellulose, natural and synthetic gums, gelatin, pregelatinized starch, polyvinylpyrrolidone, cellulose derivatives, dibasic calcium phosphate, kaolin, inorganic salts, calcium carbonate, sodium bicarbonate, sodium carbonate and combinations thereof. The saccharide may be, for example, glucose, galactose, dextrose, fructose; the disaccharide may be, for example, sucrose, lactose, lactose monohydrate, maltose, trehalose, maltose; the polysaccharide may be, for example, starch, maltodextrin; and the polyalcohol may be, for example, sorbitol, xylitol, inositol, lactitol, mannitol, spray-dried mannitol. In some embodiments, the diluent is microcrystalline cellulose, lactose monohydrate or a combination thereof.

The excipients may comprise a lubricant. In some embodiments, the lubricant comprises magnesium stearate, calcium stearate, glyceryl behenate, sodium stearyl fumarate, stearic acid, talc, zinc stearate and mixtures thereof.

In certain embodiments, the dosage forms comprise 4-7 wt. % of deutetrabenazine relative to the total weight of the dosage form; 20-40 wt. % of crospovidone relative to the total weight of the dosage form; 20-50 wt. % of hydroxypropyl methylcellulose relative to the total weight of the dosage form; 15-45 wt. % of a polyacrylic acid polymer relative to the total weight of the dosage form; and 0.3-2 wt. % of magnesium stearate relative to the total weight of the dosage form.

In certain embodiments, the dosage forms comprise 4-7 wt. % of deutetrabenazine relative to the total weight of the dosage form; 10-30 wt. % of copovidone relative to the total weight of the dosage form; 20-40 wt. % of crospovidone relative to the total weight of the dosage form; 20-50 wt. % of hydroxypropyl methylcellulose relative to the total weight of the dosage form; 2-15 wt. % of a polyacrylic acid polymer relative to the total weight of the dosage form; and 0.3-2 wt. % of magnesium stearate relative to the total weight of the dosage form.

In certain embodiments, the dosage forms comprise 4-7 wt. % of deutetrabenazine relative to the total weight of the dosage form; 5-20 wt. % of microcrystalline cellulose relative to the total weight of the dosage form; 10-30 wt. % of copovidone relative to the total weight of the dosage form; 20-40 wt. % of crospovidone relative to the total weight of the dosage form; 20-50 wt. % of hydroxypropyl methylcellulose relative to the total weight of the dosage form; 2-15 wt. % of a polyacrylic acid polymer relative to the total weight of the dosage form; and 0.3-2 wt. % of magnesium stearate relative to the total weight of the dosage form.

In certain embodiments, the dosage forms comprise 4-7 wt. % of deutetrabenazine relative to the total weight of the dosage form; 0.2-0.5 wt. % of a mixture of butylated hydroxyanisole and butylated hydroxytoluene relative to the total weight of the dosage form; 20-50 wt. % of microcrystalline cellulose relative to the total weight of the dosage form; 4-8 wt. % of copovidone relative to the total weight of the dosage form; 20-40 wt. % of crospovidone relative to the total weight of the dosage form; 5-20 wt. % of hydroxypropyl methylcellulose relative to the total weight of the dosage form; 2-15 wt % of a polyacrylic acid polymer relative to the total weight of the dosage form; and 0.3-2 wt. % of magnesium stearate relative to the total weight of the dosage form.

In certain embodiments, the dosage forms comprises 4-7 wt. % of deutetrabenazine relative to the total weight of the dosage form; 0.2-0.5 wt. % of a mixture of butylated hydroxyanisole and butylated hydroxytoluene relative to the total weight of the dosage form; 20-50 wt. % of microcrystalline cellulose relative to the total weight of the dosage form; 4-8 wt. % of copovidone relative to the total weight of the dosage form; 20-40 wt. % of crospovidone relative to the total weight of the dosage form; 5-20 wt. % of hydroxypropyl methylcellulose relative to the total weight of the dosage form; 2-15 wt. % of a polyacrylic acid polymer relative to the total weight of the dosage form; 0.1-0.7 wt. % of sodium bicarbonate relative to the total weight of the dosage form; and 0.3-2 wt. % of magnesium stearate relative to the total weight of the dosage form.

It is now related that the dosage forms perform as disclosed when the deutetrabenazine has a median particle size of 1 to 30 micron (μm), or 1 to 20 micron, or 1 to 10 micron, 1 to 9 micron, 1 to 8 micron or about 1 to about 7 micron. The desired median particle size may be generated by, for example, milling the drug substance to low micrometer sizes. In some embodiments, the deutetrabenazine has a particle size distribution characterized by a D₉₀ of about 30 micron. In some embodiments, the deutetrabenazine has a particle size distribution characterized by a D₅₀ of about 10 micron. In some embodiments, the deutetrabenazine has a particle size distribution characterized by a D₁₀ of about 1 micron. In some embodiments, the deutetrabenazine has a particle size distribution characterized by a D₉₀ of about 30 micron, a D₅₀ of about 10 micron and a D₁₀ of about 1 micron.

The dosage forms may include a 4-7 wt. % deutetrabenazine, relative to the total weight of the dosage form. The dosage forms may include a total of 6 mg-72 mg of deutetrabenazine. In some embodiment, the dosage forms may include a total of 6 mg to about 48 mg. In some embodiments, the dosage forms comprise a total of 6 mg, or 12 mg, or 18 mg, or 24 mg, or 30 mg, or 36 mg, or 42 mg or 48 mg of deutetrabenazine.

Further provided herein are methods useful in treating VMAT2 mediated disorders. In some embodiments, the methods of treating a VMAT2 mediated disorder comprise orally administering to a patient in a need thereof, controlled release dosage forms disclosed herein. The VMAT2 mediated disorder may be a hyperkinetic movement disorder. The hyperkinetic movement disorder may be a chronic disorder, for example dystonia, dyskinesia, Huntington's disease, tardive dyskinesia, and dyskinesia in cerebral palsy. In some embodiments, the methods are effective in treating chorea associated with Huntington's disease. In some embodiments, the methods are effective in treating tardive dyskinesia. The subjects afflicted with tardive dyskinesia may be concurrently administered an antipsychotic agent. In some embodiments, the methods are effective in treating dyskinesia in cerebral palsy.

In certain embodiments, the dosage forms according to any one of the embodiments disclosed herein, is administered with food.

In certain embodiments, the dosage forms according to any one of the embodiments disclosed herein, is administered under fasting conditions.

The plasma profiles of the dosage forms following administration are favorable for once a day (“qd”) administration. In one embodiment, a single dose administration of an oral dosage form comprising 6 mg of deutetrabenazine provides an in vivo plasma profile for total α- and β-dihydrodeutetrabenazine that includes a geometric mean AUC_{0-inf} of about 90,000 to 142,750 h*pg/mL and/or a geometric mean Cₘₐₓ of less than about 4,600 pg/mL.

In one embodiment, a single dose administration of a disclosed oral dosage form comprising 12 mg of deutetrabenazine provides an in vivo plasma profile for total α- and β-dihydrodeutetrabenazine that includes a geometric mean AUC_{0-inf} of about 180,000 to 285,500 h*pg/mL and/or a geometric mean Cₘₐₓ of less than about 9,200 pg/mL.

In one embodiment, a single dose administration of a disclosed oral dosage form comprising 24 mg of deutetrabenazine provides an in vivo plasma profile for total α- and β-dihydrodeutetrabenazine that includes a geometric mean AUC_{0-inf} of about 360,000 to 571,000 h*pg/mL and/or a geometric mean Cₘₐₓ of less than about 18,400 pg/mL.

In one embodiment, a single dose administration of a disclosed oral dosage form comprising 36 mg of deutetrabenazine provides an in vivo plasma profile for total α- and β-dihydrodeutetrabenazine that includes a geometric mean AUC_{0-inf} of about 540,000 to 856,500 h*pg/mL and/or a geometric mean Cₘₐₓ of less than about 27,600 pg/mL.

In one embodiment, a single dose administration of a disclosed oral dosage form comprising 48 mg of deutetrabenazine provides an in vivo plasma profile for total α- and β-dihydrodeutetrabenazine that includes a geometric mean AUC_{0-inf} of about 720,000 to 1,142,000 h*pg/mL and/or a geometric mean Cₘₐₓ of less than about 36.800 pg/mL.

In one embodiment, administration of a disclosed oral dosage form comprising 6 mg of deutetrabenazine provides an in vivo plasma profile for total α- and β-dihydrodeutetrabenazine that includes a geometric mean AUC₀₋₂₄ of about 102,500 to 200,000 h*pg/mL at steady state and/or a mean Cₘₐₓ of less than about 10,000 pg/mL at steady state.

In one embodiment, administration of a disclosed oral dosage form comprising 12 mg of deutetrabenazine provides an in vivo plasma profile for total α- and β-dihydrodeutetrabenazine that includes a geometric mean AUC₀₋₂₄ of about 205,000 to 400,000 h*pg/mL at steady state and/or a mean Cₘₐₓ of less than about 20,000 pg/mL at steady state.

In one embodiment, administration of a disclosed oral dosage form comprising 24 mg of deutetrabenazine provides an in vivo plasma profile for total α- and β-dihydrodeutetrabenazine that includes a geometric mean AUC₀₋₂₄ of about 400,000 to 800,000 h*pg/mL at steady state and/or a mean Cₘₐₓ of less than about 40,000 pg/mL at steady state.

In one embodiment, administration of a disclosed oral dosage form comprising 36 mg of deutetrabenazine provides an in vivo plasma profile for total α- and β-dihydrodeutetrabenazine that includes a geometric mean AUC₀₋₂₄ of about 615,000 to 1,200,000 h*pg/mL at steady state and/or a mean Cₘₐₓ of less than about 60,000 pg/mL at steady state.

In one embodiment, administration of a disclosed oral dosage form comprising 48 mg of deutetrabenazine provides an in vivo plasma profile for total α- and β-dihydrodeutetrabenazine that includes a geometric mean AUC₀₋₂₄ of about 800,000 to 1,600,000 h*pg/mL at steady state and/or a mean Cₘₐₓ of less than about 80,000 pg/mL at steady state.

In one embodiment, the invention provides methods of treating hyperkinetic movement disorder in a subject in need thereof comprising orally administering to the subject a once daily gastro retentive dosage form according to any one of the embodiments of the invention wherein single dose administration of the gastro retentive dosage form, which comprises a total amount of 6 mg of deutetrabenazine, provides an in vivo plasma profile for total α- and β-dihydrodeutetrabenazine that includes a geometric mean AUC_{0-inf} of about 90,000 to 142,750 h*pg/mL.

In one embodiment, the invention provides methods of treating hyperkinetic movement disorder in a subject in need thereof comprising orally administering to the subject a once daily gastro retentive dosage form according to any one of the embodiments of the invention wherein single dose administration of the gastro retentive dosage form, which comprises a total amount of 6 mg of deutetrabenazine, provides an in vivo plasma profile for total α- and β-dihydrodeutetrabenazine that includes a geometric mean Cₘₐₓ of less than about 4,600 pg/mL.

In one embodiment, the invention provides methods of treating a hyperkinetic movement disorder in a subject in need thereof comprising orally administering to the subject a once daily gastro retentive dosage form according to any one of the embodiments of the invention wherein single dose administration of the gastro retentive dosage form, which comprises a total amount of 12 mg of deutetrabenazine, provides an in vivo plasma profile for total α- and β-dihydrodeutetrabenazine that includes a geometric mean AUC_{0-inf} of about 180,00 to 285,500 h*pg/mL.

In one embodiment, the invention provides methods of treating a hyperkinetic movement disorder in a subject in need thereof comprising orally administering to the subject a once daily gastro retentive dosage form according to any one of the embodiments of the invention wherein single dose administration of the gastro retentive dosage form, which comprises a total amount of 12 mg of deutetrabenazine, provides an in vivo plasma profile for total α- and β-dihydrodeutetrabenazine that includes a geometric mean Cₘₐₓ of less than about 9,200 pg/mL.

In one embodiment, the invention provides methods of treating a hyperkinetic movement disorder in a subject in need thereof comprising orally administering to the subject a once daily gastro retentive dosage form according to any one of the embodiments of the invention wherein single dose administration of the gastro retentive dosage form, which comprises a total amount of 24 mg of deutetrabenazine, provides an in vivo plasma profile for total α- and β-dihydrodeutetrabenazine that includes a geometric mean AUC_{0-inf} of about 360,000 to 571,000 h*pg/mL.

In one embodiment, the invention provides methods of treating a hyperkinetic movement disorder in a subject in need thereof comprising orally administering to the subject a once daily gastro retentive dosage form according to any one of the embodiments of the invention wherein single dose administration of the gastro retentive dosage form, which comprises a total amount of 24 mg of deutetrabenazine, provides an in vivo plasma profile for total α- and β-dihydrodeutetrabenazine that includes a geometric mean Cₘₐₓ of less than about 18,400 pg/mL.

In one embodiment, the invention provides methods of treating a hyperkinetic movement disorder in a subject in need thereof comprising orally administering to the subject a once daily gastro retentive dosage form according to any one of the embodiments of the invention wherein single dose administration of the gastro retentive dosage form, which comprises a total amount of 36 mg of deutetrabenazine, provides an in vivo plasma profile for total α- and β-dihydrodeutetrabenazine that includes a geometric mean AUC_{0-inf} of about 540,000 to 856,500 h*pg/mL.

In one embodiment, the invention provides methods of treating a hyperkinetic movement disorder in a subject in need thereof comprising orally administering to the subject a once daily gastro retentive dosage form according to any one of the embodiments of the invention wherein single dose administration of the gastro retentive dosage form, which comprises a total amount of 36 mg of deutetrabenazine, provides an in vivo plasma profile for total α- and β-dihydrodeutetrabenazine that includes a geometric mean Cₘₐₓ of less than about 27,600 pg/mL.

In one embodiment, the invention provides methods of treating a hyperkinetic movement disorder in a subject in need thereof comprising orally administering to the subject a once daily gastro retentive dosage form according to any one of the embodiments of the invention wherein single dose administration of the gastro retentive dosage form, which comprises a total amount of 48 mg of deutetrabenazine, provides an in vivo plasma profile for total α- and β-dihydrodeutetrabenazine that includes a geometric mean AUC_{0-inf} of about 720,000 to 1,142,000 h*pg/mL.

In one embodiment, the invention provides methods of treating a hyperkinetic movement disorder in a subject in need thereof comprising orally administering to the subject a once daily gastro retentive dosage form according to any one of the embodiments of the invention wherein single dose administration of the gastro retentive dosage form, which comprises a total amount of 48 mg of deutetrabenazine, provides an in vivo plasma profile for total α- and β-dihydrodeutetrabenazine that includes a geometric mean Cₘₐₓ of less than about 36,800 pg/mL.

In one embodiment, the invention provides methods of treating a hyperkinetic movement disorder in a subject in need thereof comprising orally administering to the subject a once daily gastro retentive dosage form according to any one of the embodiments of the invention wherein the gastro retentive dosage form which comprises a total amount of 6 mg of deutetrabenazine, provides an in vivo plasma profile for total α- and β-dihydrodeutetrabenazine at steady state that includes a mean AUC₀₋₂₄ of about 102,500 to 200,000 h*pg/mL.

In one embodiment, the invention provides methods of treating a hyperkinetic movement disorder in a subject in need thereof comprising orally administering to the subject a once daily gastro retentive dosage form according to any one of the embodiments of the invention wherein the gastro retentive dosage form which comprises a total amount of 6 mg of deutetrabenazine provides an in vivo plasma profile for total α- and β-dihydrodeutetrabenazine at steady state that includes a mean Cₘₐₓ of less than about 10,000 pg/mL.

In one embodiment, the invention provides methods of treating a hyperkinetic movement disorder in a subject in need thereof comprising orally administering to the subject a once daily gastro retentive dosage form according to any one of the embodiments of the invention wherein the gastro retentive dosage form which comprises a total amount of 12 mg of deutetrabenazine provides an in vivo plasma profile for total α- and β-dihydrodeutetrabenazine at steady state that includes a mean AUC₀₋₂₄ of about 205,000 to 400,000 h*pg/mL.

In one embodiment, the invention provides methods of treating a hyperkinetic movement disorder in a subject in need thereof comprising orally administering to the subject a once daily gastro retentive dosage form according to any one of the embodiments of the invention wherein the gastro retentive dosage form which comprises a total amount of 12 mg of deutetrabenazine, provides an in vivo plasma profile for total α- and β-dihydrodeutetrabenazine at steady state that includes a mean Cₘₐₓ of less than about 20,000 pg/mL.

In one embodiment, the invention provides methods of treating a hyperkinetic movement disorder in a subject in need thereof comprising orally administering to the subject a once daily gastro retentive dosage form according to any one of the embodiments of the invention wherein the gastro retentive dosage form which comprises a total amount of 24 mg of deutetrabenazine, provides an in vivo plasma profile for total α- and β-dihydrodeutetrabenazine at steady state that includes a mean AUC₀₋₂₄ of about 410,000 to 800,000 h*pg/mL.

In one embodiment, the invention provides methods of treating a hyperkinetic movement disorder in a subject in need thereof comprising orally administering to the subject a once daily gastro retentive dosage form according to any one of the embodiments of the invention wherein the gastro retentive dosage form which comprises a total amount of 24 mg of deutetrabenazine, provides an in vivo plasma profile for total α- and β-dihydrodeutetrabenazine at steady state that includes a mean Cₘₐₓ of less than about 40,000 pg/mL.

In one embodiment, the invention provides methods of treating a hyperkinetic movement disorder in a subject in need thereof comprising orally administering to the subject a once daily gastro retentive dosage form according to any one of the embodiments of the invention wherein the gastro retentive dosage form which comprises a total amount of 36 mg of deutetrabenazine, provides an in vivo plasma profile for total α- and β-dihydrodeutetrabenazine at steady state that includes a mean AUC₀₋₂₄ of about 615,000 to 1,200,000 h*pg/mL.

In one embodiment, the invention provides methods of treating a hyperkinetic movement disorder in a subject in need thereof comprising orally administering to the subject a once daily gastro retentive dosage form according to any one of the embodiments of the invention wherein the gastro retentive dosage form which comprises a total amount of 36 mg of deutetrabenazine, provides an in vivo plasma profile for total α- and β-dihydrodeutetrabenazine at steady state that includes a mean Cₘₐₓ of less than about 60,000 pg/mL.

In one embodiment, the invention provides methods of treating a hyperkinetic movement disorder in a subject in need thereof comprising orally administering to the subject a once daily gastro retentive dosage form according to any one of the embodiments of the invention wherein the gastro retentive dosage form which comprises a total amount of 48 mg of deutetrabenazine, provides an in vivo plasma profile for total α- and β-dihydrodeutetrabenazine at steady state that includes a mean AUC₀₋₂₄ of about 820,000 to 1,600,000 h*pg/mL.

In one embodiment, the invention provides methods of treating a hyperkinetic movement disorder in a subject in need thereof comprising orally administering to the subject a once daily gastro retentive dosage form according to any one of the embodiments of the invention wherein the gastro retentive dosage form which comprises a total amount of 48 mg of deutetrabenazine, provides an in vivo plasma profile for total α- and β-dihydrodeutetrabenazine at steady state that includes a mean Cₘₐₓ of less than about 80,000 pg/mL.

In one embodiment, following the administration of the gastro retentive dosage form according to the embodiments described herein, not more than 40% of the deutetrabenazine is released after 2 hours when tested in 500 mL acid phosphate buffer at pH 3.0 using a USP II dissolution apparatus.

In one embodiment, following the administration of the gastro retentive dosage form according to the embodiments described herein, not more than 20% of the deutetrabenazine is released after 2 hours when tested in 500 mL acid phosphate buffer at pH 3.0 using a USP II dissolution apparatus.

In one embodiment, following the administration of the gastro retentive dosage form according to the embodiments described herein, not more than 80% of the deutetrabenazine is released after 8 hours when tested in 500 mL acid phosphate buffer at pH 3.0 using a USP II dissolution apparatus.

In one embodiment, following the administration of the gastro retentive dosage form according to the embodiments described herein, not more than 60% of the deutetrabenazine is released after 8 hours when tested in 500 mL acid phosphate buffer at pH 3.0 using a USP II dissolution apparatus.

In one embodiment, following the administration of the gastro retentive dosage form according to the embodiments described herein, 40%-60% of the deutetrabenazine is released after 7 hours when tested in 500 mL acid phosphate buffer at pH 3.0 using a USP II dissolution apparatus.

In one embodiment, following the administration of the gastro retentive dosage form according to the embodiments described herein, about 50% of the deutetrabenazine is released after 7 hours when tested in 500 mL acid phosphate buffer at pH 3.0 using a USP II dissolution apparatus.

Further provided herein is a process for manufacturing the gastro retentive dosage forms described herein, comprising dry and direct blending of a mixture of deutetrabenazine with at least two control release polymers, each independently having a viscosity of 2,000 cPs to about 80,000 cPs and pharmaceutically acceptable excipients. The dry blend can be further compressed into monolayered tablets.

Further provided herein is a process for manufacturing the gastro retentive dosage forms described herein, comprising dry granulation of deutetrabenazine with at least two control release polymers, each independently having a viscosity of 2,000 cPs to about 80,000 cPs and pharmaceutically acceptable excipients. The granulate can be further compressed into monolayered tablets.

Further provided herein is micronized deutetrabenazine having a median particle size of 1 to 30 micron (μm), or 1 to 20 micron, or 1 to 10 micron, 1 to 9 micron, 1 to 8 micron or about 1 to about 7 micron. In some embodiments, the deutetrabenazine has a particle size distribution characterized by a D90 of about 30 micron, a D50 of about 10 micron and a D10 of about 1 micron.

### EXAMPLES

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the disclosure.

### Example 1—Manufacturing Process Development

The manufacturing process for the gastro retentive dosage form includes the following steps:
a. Manufacturing of a deutetrabenazine blend;
b. Compression of tablets;
c. Optional packaging

### Drug Substance Physical Characterization

The deutetrabenazine particle size distributions following manufacture (untreated), milling (quadro co-mill) and micronization (air jet mill) are shown in Table 1.

TABLE 1
Particle Size Distribution (PSD) of
unmilled and milled drug substance

| PSD | unmilled | co-milled | micro-milled |
|---|---|---|---|
| D10 (μm) | 9.08 | 6.11 | 1.08 |
| D50 (μm) | 59.66 | 33.55 | 3.31 |
| D90 (μm) | 213.07 | 131.55 | 7.05 |

For dry measurement of the milled and unmilled deutetrabenazine the following settings are preferably used:

| Analysis model | Mie |
|---|---|
| Obscuration | 1.12% |
| Sample measurement time | 24 sec |

Deutetrabenazine is a weakly basic compound with relatively good solubility in acidic environment (pH<4) and poorly soluble at pH ≥4 (<2.3 mg/mL). Deutetrabenazine is permeable through the small intestinal (SI) segments in a rat perfusion model. In humans, approximately 80% of a radioactive dose was recovered in urine in a human [¹⁴C]-AME study with deutetrabenazine dosed as powder in capsule (PIC) suggesting that the compound is well-absorbed in the small intestine and large intestine/colon. Levels of absorption of deutetrabenazine in the lower GI in rats was shown to be Jejunum<Mid-small intestine<Colon<Ileum, with ileum absorption about 0.0006 cm/sec.

The dosage forms disclosed herein achieve comparable pharmacokinetics (PK) of 2 daily doses (bid) of AUSTEDO® 12 mg tablets with a single daily dose (qd). Drug solubility at pH greater >3 was tested in un-milled drug substance (DS) batches, compared to co-milled and micro-milled DS batches.

For all three batches, the deutetrabenazine was dry blended with release controlling polymers and a lubricant. For samples 1, 2 and 3, un-milled deutetrabenazine, deutetrabenazine with a particle size of D90<150 micron, and deutetrabenazine with a particle size of D90<micron were used, respectively. All three blends were compressed into tablets. Table 2 shows compositions of the deutetrabenazine tablets of Samples 1, 2 and 3.

In all examples infra, the dissolution of the dosage forms was performed in 500 mL acid buffer pH 3, USP-II apparatus, 75 rpm for up to 14 hours. Samples were collected at 1, 2, 3, 4, 5, 6, 8, 10, 12 and 14 hours.

TABLE 2
Composition of Samples 1, 2 and 3.

| | Sample 1 | Sample 2 | Sample 3 |
|---|---|---|---|
| deutetrabenazine (un-milled) | 5.69% | | |
| deutetrabenazine (co-milled) | | 5.69% | |
| deutetrabenazine (micro-milled) | | | 5.69% |
| Kollidon ® SR | 23.22% | 23.22% | 23.22% |
| Crospovidone, NF | 24.88% | 24.88% | 24.88% |
| (Polyplasdone ® XL-10) | | | |
| Hypromellose 2208, USP | 35.55% | 35.55% | 35.55% |
| Carbopol ® 71G, NF | 10.07% | 10.07% | 10.07% |
| Magnesium Stearate, NF | 0.59% | 0.59% | 0.59% |

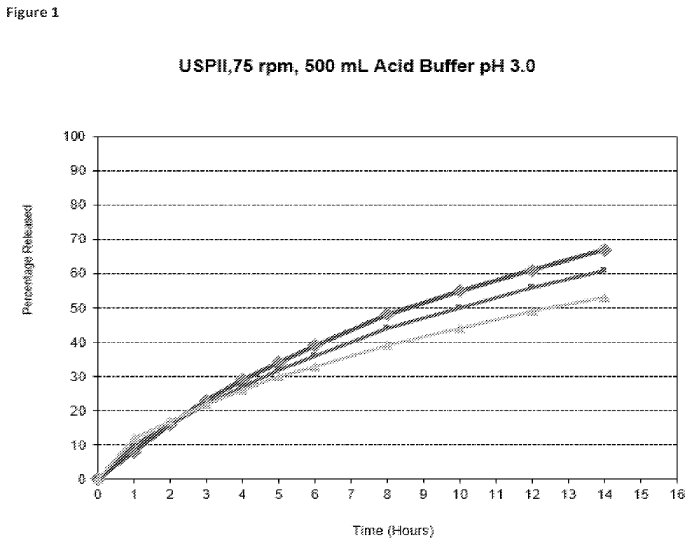

FIG. 1 shows the dissolution profiles of Samples 1, 2, and 3. The co-milled and micro-milled particles exhibit a lower dissolution profile in pH 3 than un-milled sample. The triangles represent release profile of the micro-milled sample, showing slow release (˜50% after 14 hr); the squares represent the release profile of the co-milled deutetrabenazine and the diamonds represent the release profile of the unmilled deutetrabenazine. Without being bound to any theory, it is believed that the lower dissolution profile of the finer particles can be attributed to a better mix with the control release polymers.

### Example 2—Controlled Release Gastro Retentive Solid Oral Dosage Forms

A. Dry Direct Blend

Deutetrabenazine particle size was reduced to a micrometer size (<10 micron) using an air jet milling process. The milled deutetrabenazine was dry blended with insoluble and water soluble polymers. A lubricant was screened and further mixed with the blend. The blend was compressed on a rotary tablet press. Additional sample dosage forms were manufactured and tested for dissolution.

The composition of Samples 4-7 is provided below, in Table 3.

TABLE 3

| Ingredients | Sample 4 | Sample 5 | Sample 6 | Sample 7 |
|---|---|---|---|---|
| Deutetrabenazine | 5.69% | 5.69% | 5.69% | 5.69% |
| Kollidon ® SR | 18.74% | 14.06% | 15.93% | 25.79% |
| Crospovidone, NF | 24.88% | 24.89% | 24.89% | 24.89% |
| (Polyplasdone ® XL-10) | | | | |
| Hypromellose 2208, USP | 35.54% | 35.55% | 35.55% | 35.55% |
| Carbopol ® 71G, NF | 14.57% | 19.22% | 17.35% | 7.49% |
| Magnesium Stearate, NF | 0.59% | 0.59% | 0.59% | 0.59% |

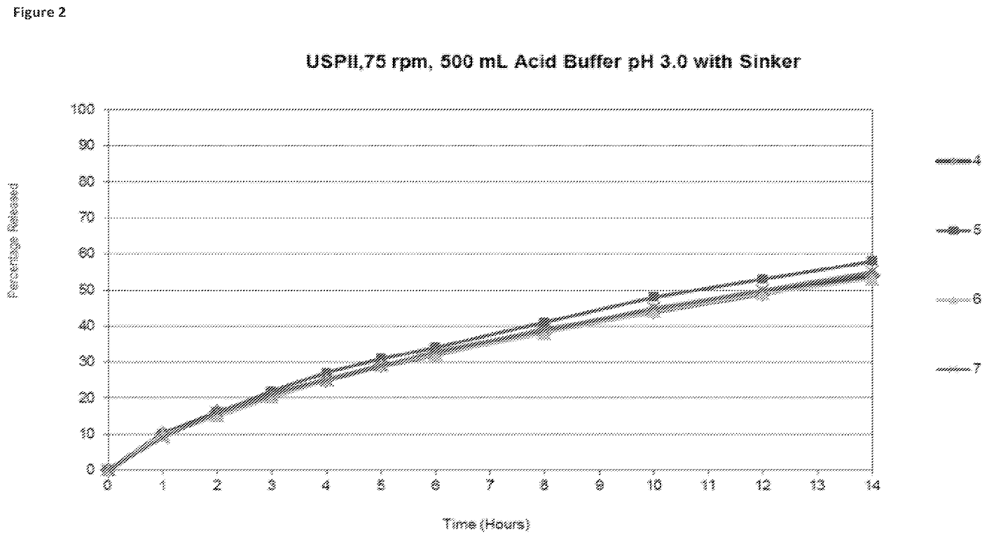

The drug release profile of samples 4-7 is provided as a graph in FIG. 2.

The compositions of Samples 8-9 are provided in Table 4.

TABLE 4

| Ingredients | Sample 8 | Sample 9 |
|---|---|---|
| Deutetrabenazine | 5.68% | 5.68% |
| Microcrystalline Cellulose | 8.51% | 14.43% |
| Kollidon ® SR | 20.34% | 18.36% |
| Crospovidone, NF | 24.83% | 24.84% |
| (Polyplasdone ® XL-10) | | |
| Hypromellose 2208, USP | 31.22% | 28.15% |
| Carbopol ® 71G, NF | 8.82% | 7.95% |
| Magnesium Stearate, NF | 0.59% | 0.59% |

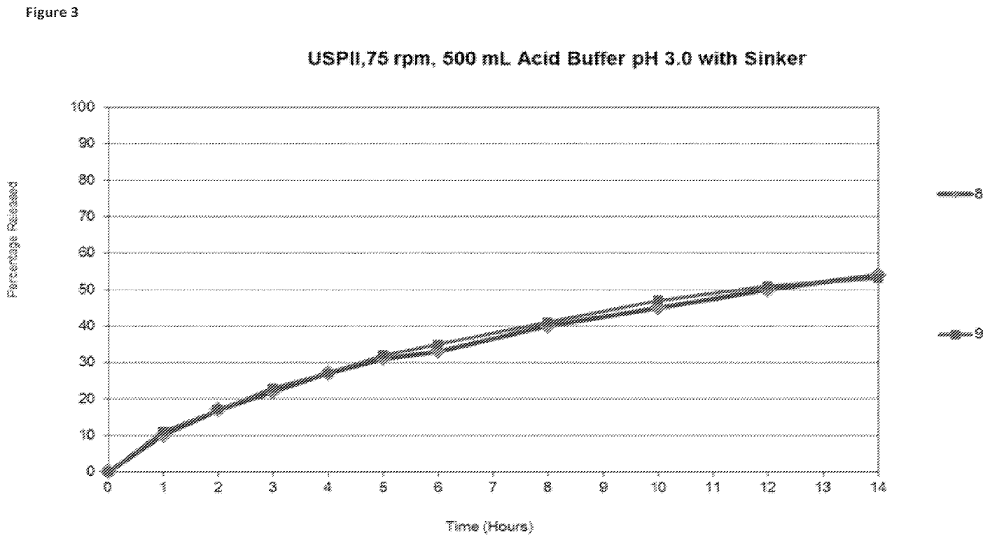

The drug release profile of samples 8-9 is provided as a graph in FIG. 3.

The compositions of Samples 10-11 are provided in Table 5.

TABLE 5

| Ingredients | Sample 10 | Sample 11 |
|---|---|---|
| Deutetrabenazine | 5.68% | 5.68% |
| Kollidon ® SR | 8.53% | 8.53% |
| Crospovidone, NF | 46.66% | 24.87% |
| (Polyplasdone ® XL-10) | | |
| Hypromellose 2208, USP | 13.03% | 13.03% |
| Hypromellose 2208, USP | 21.79% | 43.58% |
| Carbopol ® 71G, NF | 3.72% | 3.72% |
| Magnesium Stearate, NF | 0.59% | 0.59% |

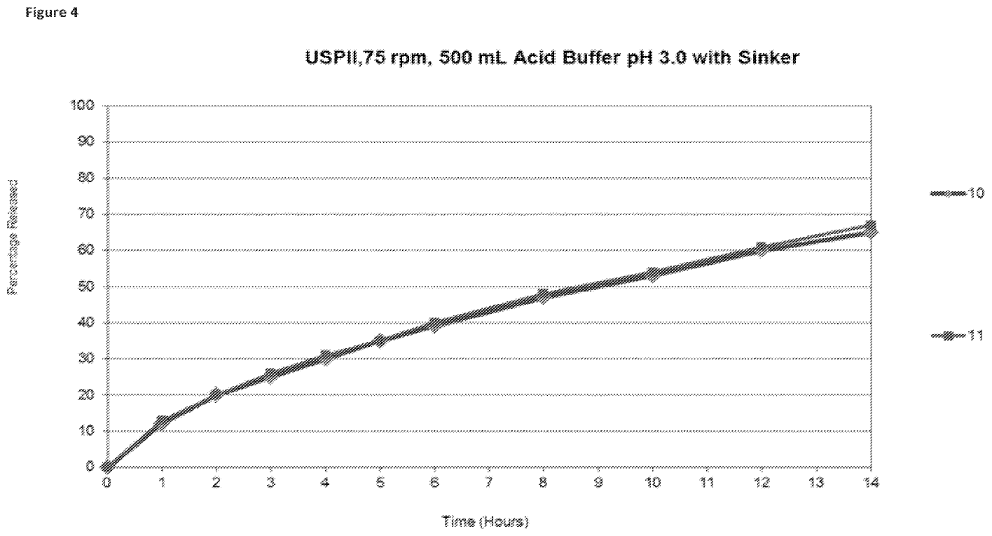

The drug release profile of samples 10-11 is provided as a graph in FIG. 4.

The compositions of Samples 12-13 are provided in Table 6.

TABLE 6

| Ingredients | Sample 12 | Sample 13 |
|---|---|---|
| Deutetrabenazine | 5.69% | 5.69% |
| Kollidon ® SR | 11.61% | 11.62% |
| Crospovidone, NF | 24.88% | 12.38% |
| (Polyplasdone ® XL-10) | | |
| Hypromellose 2208, USP | 52.18% | 64.67% |
| Carbopol ® 71G, NF | 5.05% | 5.05% |
| Magnesium Stearate, NF | 0.59% | 0.59% |

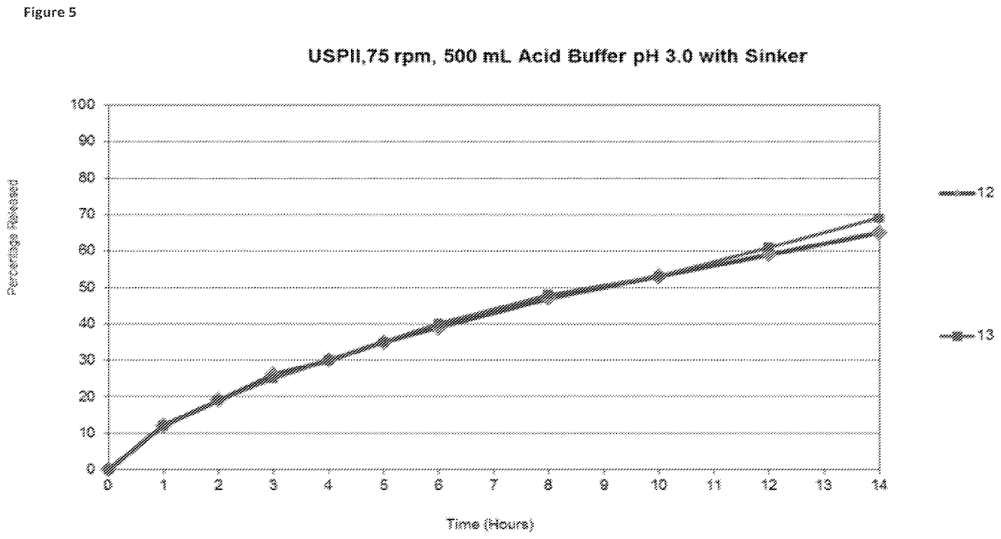

The drug release profile of samples 12-13 is provided as a graph in FIG. 5.

The following samples were also produced in a dry blend process, with the addition of antioxidants into the dry mix.

The compositions of Samples 14-17 are provided in Table 7.

TABLE 7

| Ingredients | Sample 14 | Sample 15 | Sample 16 | Sample 17 |
|---|---|---|---|---|
| Deutetrabenazine | 5.71% | 5.69% | 5.69% | 4.64% |
| Butylated Hydroxyanisole | 0.24% | 0.24% | 0.24% | 0.19% |
| Butylated Hydroxytoluene | 0.10% | 0.09% | 0.09% | 0.08% |
| Silicified Microcrystalline Cellulose, | 27.48% | 48.22% | 41.14% | 33.58% |
| NF (Prosolv ® SMCC 50) | | | | |
| Kollidon ® SR | 4.99% | 6.87% | 6.87% | 5.61% |
| Crospovidone, NF (Polyplasdone ® XL-10) | 35.66% | 24.88% | 24.88% | 38.68% |
| Hypromellose 2208, USP | 15.45% | 10.43% | 10.43% | 8.51% |
| Carbopol ® 71G, NF | 10.10% | 2.99% | 10.07% | 8.22% |
| Magnesium Stearate, NF | 0.59% | 0.59% | 0.59% | 0.48% |

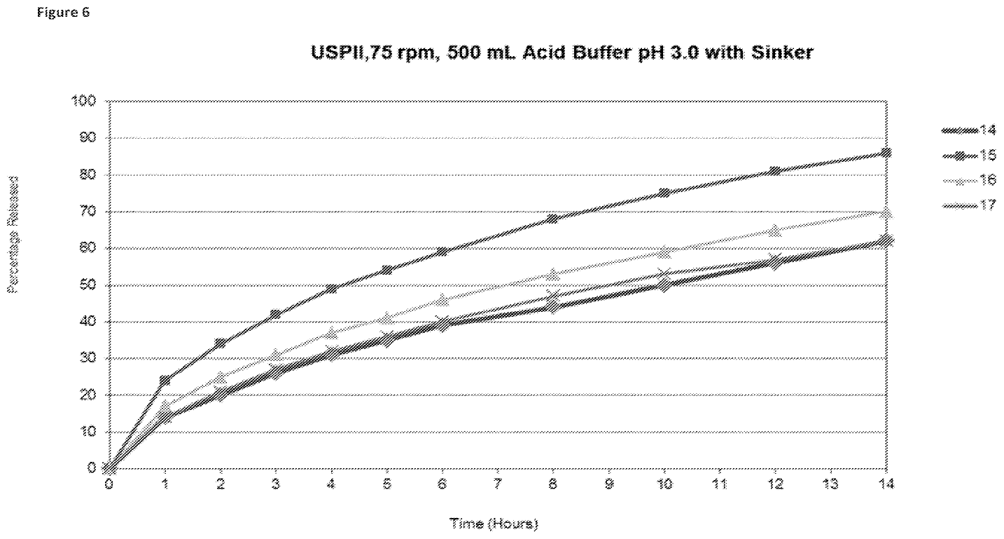

The drug release profile of samples 14-17 is provided as a graph in FIG. 6.

The compositions of Samples 18-19 are provided in Table 8.

TABLE 8

| Ingredients | Sample 18 | Sample 19 |
|---|---|---|
| Deutetrabenazine | 5.69% | 4.46% |
| Butylated Hydroxyanisole | 0.24% | 0.19% |
| Butylated Hydroxytoluene | 0.09% | 0.07% |
| Silicified Microcrystalline Cellulose, | 41.14% | 32.27% |
| NF (Prosolv ® SMCC 50) | | |
| Kollidon ® SR | 6.87% | 5.39% |
| Crospovidone, NF | 24.88% | 37.17% |
| (Polyplasdone ® XL-10) | | |
| Hypromellose 2208, USP | 10.43% | 12.08% |
| Carbopol ® 71G, NF | 10.07% | 7.90% |
| Magnesium Stearate, NF | 0.59% | 0.46% |

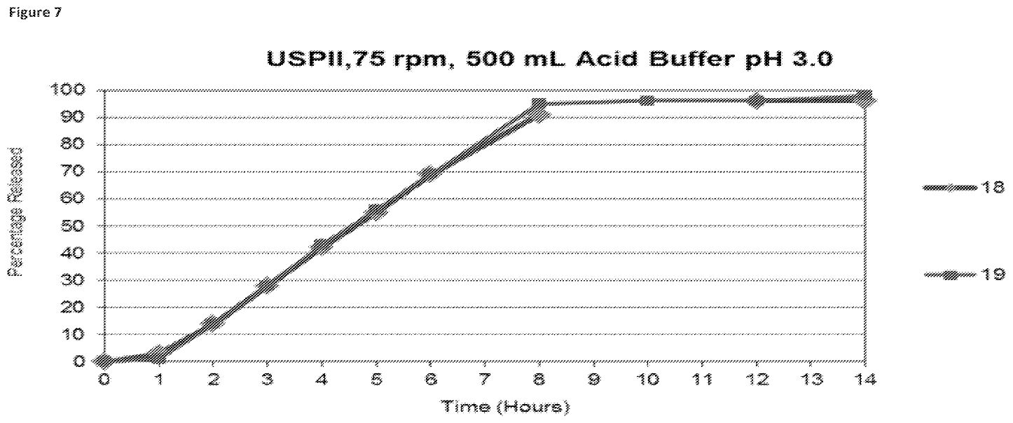

The drug release profile of samples 18-19 is provided as a graph in FIG. 7.

The composition of Samples 20-21 are provided in Table 9.

TABLE 9

| Ingredients | Sample 20 | Sample 21 |
|---|---|---|
| Deutetrabenazine (Micronized) | 5.67% | 5.66% |
| Butylated Hydroxyanisole | 0.24% | 0.24% |
| Butylated Hydroxytoluene | 0.09% | 0.09% |
| Silicified Microcrystalline Cellulose, | 41.04% | 40.94% |
| NF (Prosolv ® SMCC 50) | | |
| Sodium Bicarbonate, USP | 0.24% | 0.47% |
| Polysorbate 80 NF | 0.95% | 0.94% |
| Kollidon ® SR | 6.86% | 6.84% |
| Crospovidone, NF | 24.82% | 24.76% |
| (Polyplasdone ® XL-10) | | |
| Hypromellose 2208, USP | 10.40% | 10.38% |
| Carbopol ® 71G, NF | 10.05% | 10.02% |
| Magnesium Stearate, NF | 0.59% | 0.59% |

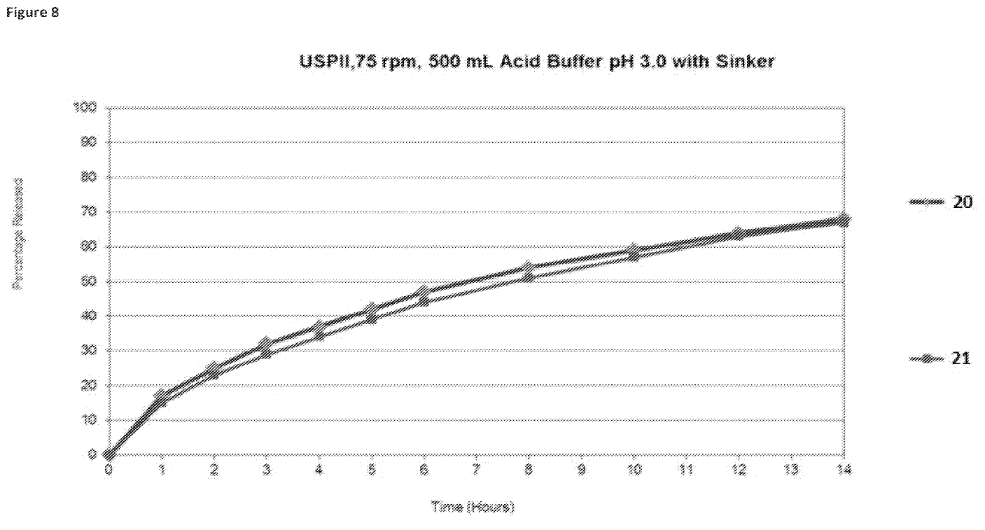

The drug release profile of samples 20-21 is provided as a graph in FIG. 8.

B. Granulation

Butylated hydroxytoluene and butylated hydroxyanisole were dissolved in alcohol USP. Micronized active, silicified microcrystalline cellulose, NF (Prosolv® SMCC 50) and sodium bicarbonate. USP (for Sample 23) were screened through #30 mesh and mixed for 5 minutes using GMX-5. Dry blend was granulated using butylated hydroxyanisole and butylated hydroxytoluene solution. Granules were dried in tray dryer oven at 60° C. to get LOD below 2%. Granules were screened through #20 mesh. Screened granules were blended with Kollidon® SR, Crospovidone, NF (Polyplasdone® XL-10), Hypromellose 2208, USP and Carbopol® 71G, NF after screening through #20 mesh for 15 minutes and then lubricated for 5 minutes with Magnesium Stearate (#30 mesh screened). Blend was compressed using 0.5512″×0.3937″ diamond shaped punches.

The compositions of Samples 22-23 are provided in Table 10.

TABLE 10

| Ingredients | Sample 22 | Sample 23 |
|---|---|---|
| Deutetrabenazine | 5.69% | 5.14% |
| Butylated Hydroxyanisole | 0.24% | 0.21% |
| Butylated Hydroxytoluene | 0.09% | 0.09% |
| Silicified Microcrystalline Cellulose, | 41.14% | 37.17% |
| NF (Prosolv ® SMCC 50) | | |
| Sodium Bicarbonate, USP | — | 9.64% |
| Kollidon ® SR | 6.87% | 6.21% |
| Crospovidone, NF | 24.88% | 22.48% |
| (Polyplasdone ® XL-10) | | |
| Hypromellose 2208, USP | 10.43% | 9.42% |
| Carbopol ® 71G, NF | 10.07% | 9.10% |
| Magnesium Stearate, NF | 0.59% | 0.54% |

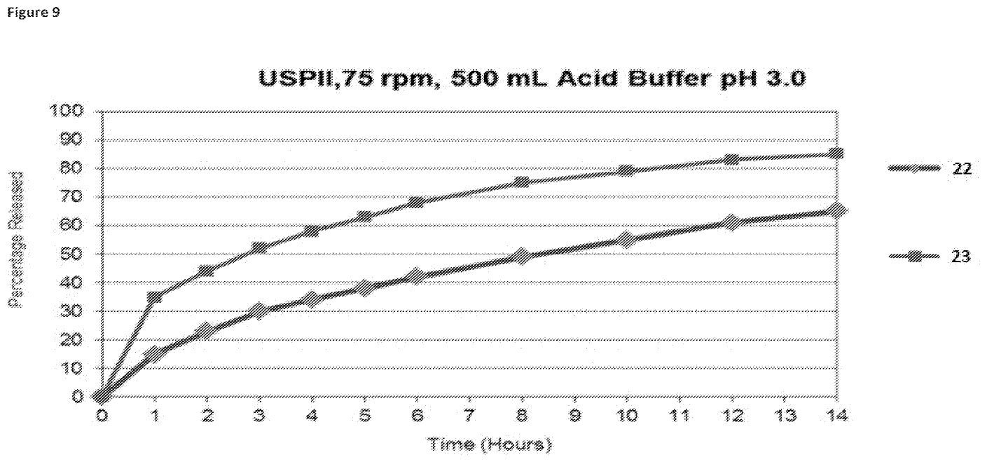

The drug release profile of samples 22-23 is provided as a graph in FIG. 9.

### Example 3—In Vivo Release Profile in Dogs

The trial was conducted as an open-label, randomized, single-dose, partial crossover comparative bioavailability study. The study was designed to determine the AUC0-t, AUC0-24, Cmax and Tmax of deutetrabenazine and total deuterated α- and β-dihydrotetrabenazine (deuHTBZ) metabolites. The sample group consisted of 4 male beagle dogs. Each dog was administered two treatments. The first treatment, treatment A, comprised of single 12 mg AUSTEDO® tablets administered twice, 8 hours apart (bid); the second treatment, treatment B, comprised of a single administration of the gastro retentive dosage form of Sample 20. A washout period of 14 days was kept before crossing over to the next treatment. Each dog was administered a single oral dose with 10 ml water. Prior to each dose administration, the dogs were pretreated with pentagastrin (to stimulate gastric acid secretion.

Blood samples were collected before dosing and at predetermined time points through 24 hours post-dosing. The samples were analyzed for deutetrabenazine and total deuHTBZ in plasma using an LC-MS/MS method. The PK profiles following oral administration of the two treatments are presented in Table 11.

TABLE 11

| Analyte | Parameter | Treatment A | Treatment B | |
|---|---|---|---|---|
| Deutetra- | Cₘₐₓ (ng/mL) | 1^{st} dose: 19.9 (16.7) | 12.6 | (5.6) |
| benazine | | 2^{nd} dose: 28.1 (20.7) | | |
| | tₘₐₓ (h)* | 1^{st} dose: 1.0 [0.5-1.5] | 1.5 | [0.5-2.0] |
| | | 2^{nd} dose: 1.5 [1.0-4.0] | | |
| | AUC₀₋ₜ | 107 (67) | 40 | (16) |
| | (ng · h/mL) | | | |
| | AUC₀₋₂₄ | 107 (66) | 43 | (16) |
| | (ng · h/mL) | | | |
| Total | Cₘₐₓ (ng/mL) | 1^{st} dose: 201 (126) | 174 | (83) |
| deuHTBZ | | 2^{nd} dose: 174 (162) | | |
| | tₘₐₓ (h)* | 1^{st} dose: 1.3 [0.5-1.5] | 1.8 | [1.5-3.0] |
| | | 2^{nd} dose: 1.5 [1.0-4.0] | | |
| | AUC₀₋ₜ | 1162 (977) | 647 | (364) |
| | (ng · h/mL) | | | |
| | AUC₀₋₂₄ | 1162 (977) | 655 | (357) |
| | (ng · h/mL) | | | |

*Values are mean (SD), except those for tmax which are median [range]

Results

The tested gastro retentive dosage form (Treatment B) was well-tolerated in dogs after single oral doses, without any instances of adverse clinical signs or emesis. The tested gastro retentive dosage form produced peak plasma concentrations (Cₘₐₓ) and total systemic exposures (AUC) of deutetrabenazine and total deuHTBZ that were lower than AUSTEDO®. These preliminary studies provided a favorable indication about the formulation's design and its behavior in vivo. The observed values, on the lower side for Cₘₐₓ and AUC, can be attributed to differences between dogs and humans (deutetrabenazine/deuHTBZ Cₘₐₓ ratio is about 10% in dogs and is <1% in humans).

### Example 4—Human Single Dose Bioavailability Assessment

Gastro retentive dosage forms containing deutetrabenazine are produced as disclosed in Example 2 and studied in a single dose pharmacokinetic study.

The primary objective is to assess the comparative bioavailability (BA) of deutetrabenazine and total deuHTBZ metabolites following a single administration of the gastro retentive dosage form (“Test”) compared to a single 12 mg AUSTEDO® tablet administered twice (“Ref”), 12 hours apart (bid), under fasted conditions.

Study Population and Number of Subjects: The study includes healthy male and female non-smoking subjects.

Duration of Subject Participation: The study includes a screening period of 2-4 weeks (period 1), an open label treatment period with the once daily gastro retentive test dosage forms (“Test”) and the reference formulation (“Ref”) (period 2), and a follow-up visit at least 1 day later (period 3).

Treatments:

Treatment sequence A:

Day 1—administration of Test.

Days 2-3—at least 6 hours wash out of Test followed by administration of Ref.

Treatment sequence B:

Day 1—administration of Ref

Days 2-3—at least 6 hours wash out of Ref, followed by administration of Test.

The primary objective was addressed using the following parameters:
maximum observed concentration (Cmax)
area under the plasma concentration-time (AUC) from time 0 to the time of the last measurable plasma concentration (AUC0-t)
AUC extrapolated to infinity (AUC0-∞)
AUC from time 0 to 24 hours post dose (AUC0-24 h)

Analyses

AUC0-t. AUC0-∞, and AUC0-24 h are calculated using the trapezoidal rule. The Cmax, AUC0-t, AUC0-∞, and AUC0-24 h data are natural log-transformed prior to the statistical analysis. Comparisons of Cmax, AUC0-t, AUC0-∞, and AUC0-24 h between treatments (Test vs Ref) will be carried out using a separate parametric analysis of variance (ANOVA) model with fixed effect terms for sequence, period, treatment group, and a random effect of subject within sequence. The difference between the Ref and the Test will be evaluated by constructing 90% confidence intervals for the Test/Ref ratios, based on the least-square means from the ANOVA for the log-transformed Cmax, AUC0-t, AUC0-∞ and AUC0-24 h. The treatment difference and the associated 90% confidence interval estimated from the ANOVA on the log scale will be back-transformed to obtain the estimated ratio of geometric means between treatment groups and the 90% confidence interval for this ratio.

### Results

The once-daily dose of Test dosage forms provide similar deuHTBZ plasma concentrations observed for the Ref. The gastro retentive dosage forms disclosed herein are administered once daily and provide a similar treatment effect to that of AUSTEDO® and also have no safety concerns.

### Example 5—Multiple Dose Bioavailability Assessment

The gastro retentive dosage forms containing 24 mg of deutetrabenazine are produced as disclosed in Example 2 and are studied in an open label, randomized, multiple-dose, 2-way crossover study in healthy volunteers.

The primary objective is to assess the bioequivalence (BE) of qd administration of Test, compared to bid administration of Ref, under fasted or fed conditions.

Treatment includes 7 days repeated dosing of Test once daily versus 7 days repeated dosing of Ref, bid.

AUCt, Cₘₐₓ, tₘₐₓ, Cₘᵢₙ, Cav for deutetrabenazine and deuHTBZ are analyzed, at steady state.

### Results

Multiple dosing of Test has comparable PK parameters to that of Ref, at steady state. Therefore similar efficacy response is expected with once daily administration, having no safety concerns.

### Example 6—Food Effect Study

Gastro retentive dosage forms containing deutetrabenazine are produced as disclosed in Example 2 and studied in an open label, randomized, two-way crossover study, to assess the comparative bioavailability of deutetrabenazine and deuHTBZ in the fed compared to the fasted state, following a single administration of 24 mg, once daily (qd) gastro retentive formulation.

Treatment includes:
A—gastro retentive formulation given as a single oral dose with water after an overnight fast of at least 10 hours.
B—gastro retentive formulation given as a single oral dose with water, 30 minutes after the start of standardized high calorie, high fat breakfast administered after an overnight fast of at least 10 hours.

Subject will receive treatments A/B with at least 6 days washout period.

AUCt, Cmax, tmax, Cmin, Cav for deutetrabenazine and deuHTBZ will be analyzed.

### Results

The similar plasma concentrations of deutetrabenazine and deuHTBZ, following single administration with or without food, show that the gastro retentive dosage from can be administered independent of food intake

All patents, patent applications, and publications mentioned in the specification are indicative of the levels of those of ordinary skill in the art to which the invention pertains. All patents, patent applications, and publications are herein incorporated by reference to the same extent as if each individual publication was specifically and individually indicated to be incorporated by reference. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

For the foregoing embodiments, each embodiment disclosed herein is contemplated as being applicable to each of the other disclosed embodiments. For instance, the elements recited in the method embodiments can be used in the pharmaceutical composition, package, and use embodiments described herein and vice versa.

### Aspects

Aspect 1. A controlled release gastro retentive solid oral dosage form for once daily administration of deutetrabenazine comprising deutetrabenazine, at least one release controlling polymer and a pharmaceutically acceptable excipient.
Aspect 2. The gastro retentive solid oral dosage form of Aspect 1, wherein the release controlling polymer comprises a water soluble polymer, a water insoluble polymer or mixtures thereof.
Aspect 3. The gastro retentive solid oral dosage form of Aspect 2, wherein the water soluble polymer comprises hydroxypropyl cellulose, hydroxypropyl methylcellulose, polyvinyl pyrrolidone, polyvinyl alcohol, a polyacrylic acid polymer or mixtures thereof.
Aspect 4. The gastro retentive solid oral dosage form of Aspect 3, wherein the water soluble polymer comprises hydroxypropyl methylcellulose, a polyacrylic acid polymer or mixtures thereof.
Aspect 5. The gastro retentive solid oral dosage form of Aspect 4, wherein the polyacrylic acid polymer comprises a cross linked polyacrylic acid polymer, such as Carbopol®.
Aspect 6. The gastro retentive solid oral dosage form of Aspect 2, wherein the water insoluble polymer comprises crospovidone, copovidone, croscarmellose sodium, starch, sodium starch glycolate, colloidal silica, silica, ethyl cellulose, polyvinyl acetate, or mixtures thereof.
Aspect 7. The gastro retentive solid oral dosage form of Aspect 6, wherein the water insoluble polymer comprises crospovidone, copovidone, or mixtures thereof.
Aspect 8. The gastro retentive solid oral dosage form of Aspect 7, wherein the copovidone is Kollidon®.
Aspect 9. The gastro retentive solid oral dosage form of any one of the preceding Aspects, comprising a release controlling polymer mixture of crospovidone, copovidone, hydroxypropyl methylcellulose and a polyacrylic acid polymer.
Aspect 10. The gastro retentive solid oral dosage form of any one of the preceding Aspects, comprising 5 to 65 wt. % of the release controlling polymer, relative to the total weight of the solid oral dosage form.
Aspect 11. The gastro retentive solid dosage form of any one of the preceding Aspects, wherein the pharmaceutically acceptable excipient comprises a diluent, a disintegrant, a binder, a gas-generating agent, an antioxidant, a lubricant or combinations thereof.
Aspect 12. The gastro retentive solid oral dosage form of Aspect 11, wherein the pharmaceutically acceptable excipient comprises a gas-generating agent and wherein the gas-generating agent comprises a carbonate salt selected from the group consisting of sodium bicarbonate, a mixture of sodium bicarbonate and citric acid, calcium carbonate, a mixture of calcium carbonate and citric acid and a mixture thereof.
Aspect 13. The gastro retentive solid oral dosage form of Aspect 12, wherein the gas-generating agent is present in the amount of 5 to 15 wt. %, relative to the total weight of the solid oral dosage form.
Aspect 14. The gastro retentive solid oral dosage form of any one of Aspects 11-13, wherein the pharmaceutically acceptable excipient comprises an anti-oxidant, and wherein the antioxidant comprises propyl gallate, 6-ethoxy-1,2-digydro-2,2,4-trimethylquinoline (ethoxyquin), nordihydroguaiaretic acid (NDGA), butylated hydroxyanisole, butylated hydroxytoluene or any mixture thereof.
Aspect 15. The gastro retentive solid oral dosage form of Aspect 14, wherein the antioxidant comprises a mixture of butylated hydroxyanisole and butylated hydroxytoluene.
Aspect 16. The gastro retentive solid oral dosage form of any one of Aspects 11-15, wherein the antioxidant is present in the amount of 0.1 to 1 wt. %, relative to the total weight of the solid oral dosage form.
Aspect 17. The gastro retentive solid oral dosage form of any one of Aspects 11-16, wherein the pharmaceutically acceptable excipient comprises a binder, and wherein the binder comprises, microcrystalline cellulose, starch, gelatin, agar, natural and synthetic gums or any mixture thereof.
Aspect 18. The gastro retentive solid oral dosage form of any one of Aspects 11-17, wherein the binder is present in the amount of 5 to 50 wt. %, relative to the total weight of the solid oral dosage form.
Aspect 19. The gastro retentive solid oral dosage form of any one of the preceding Aspects, comprising 4-7 wt. % deutetrabenazine, relative to the total weight of the dosage form.
Aspect 20. The gastro retentive solid oral dosage form of any one of the preceding Aspects, wherein the total amount of deutetrabenazine in the dosage form is from about 6 mg to about 48 mg.
Aspect 21. The gastro retentive solid oral dosage form of Aspect 20, wherein the total amount of deutetrabenazine in the dosage form is 6 mg.
Aspect 22. The gastro retentive solid oral dosage form of Aspect 20, wherein the total amount of deutetrabenazine in the dosage form is 12 mg.
Aspect 23. The gastro retentive solid oral dosage form of Aspect 20, wherein the total amount of deutetrabenazine in the dosage form is 24 mg.
Aspect 24. The gastro retentive solid oral dosage form of Aspect 20, wherein the total amount of deutetrabenazine in the dosage form is 36 mg.
Aspect 25. The gastro retentive solid oral dosage form of Aspect 20, wherein the total amount of deutetrabenazine in the dosage form is 48 mg.
Aspect 26. The gastro retentive solid oral dosage form of any one of the preceding Aspects, wherein the deutetrabenazine is micronized deutetrabenazine having a mean particle size of about 1 μm to about 30 m in diameter.
Aspect 27. The gastro retentive solid oral dosage form of Aspect 26, wherein the deutetrabenazine has a particle size distribution characterized by a D90 of 30 μm.
Aspect 28. The gastro retentive solid oral dosage form of Aspect 26, wherein the deutetrabenazine has a particle size distribution characterized by a D50 of 10 μm.
Aspect 29. The gastro retentive solid oral dosage form of Aspect 26, wherein the deutetrabenazine has a particle size distribution characterized by a D10 of 1 μm.
Aspect 30. The gastro retentive solid oral dosage form of any one of the preceding Aspects, comprising:
   a. 4-7 wt. % of deutetrabenazine,
   b. 20-40 wt. % of crospovidone,
   c. 20-50 wt. % of hydroxypropyl methylcellulose,
   d. 15-45 wt. % of microcrystalline cellulose,
   e. 0.3-2 wt. % of magnesium stearate
Aspect 31. The gastro retentive solid oral dosage form of any one of Aspects 1-29, comprising:
   a. 4-7 wt. % of deutetrabenazine,
   b. 20-40 wt. % of crospovidone,
   c. 20-50 wt. % of hydroxypropyl methylcellulose,
   d. 15-45 wt. % of a polyacrylic acid polymer,
   e. 0.3-2 wt. % of magnesium stearate
Aspect 32. The gastro retentive solid oral dosage form of any one of Aspects 1-29, comprising:
   a. 4-7 wt. % of deutetrabenazine,
   b. 10-30 wt. % of copovidone,
   c. 20-40 wt. % of crospovidone,
   d. 20-50 wt. % of hydroxypropyl methylcellulose,
   e. 2-15 wt. % of a polyacrylic acid polymer,
   f. 0.3-2 wt. % of magnesium stearate
Aspect 33. The gastro retentive solid oral dosage form of any one of Aspects 1-29, comprising:
   a. 4-7 wt. % of deutetrabenazine,
   b. 5-20 wt. % of microcrystalline cellulose
   c. 10-30 wt. % of copovidone,
   d. 20-40 wt. % of crospovidone,
   e. 20-50 wt. % of hydroxypropyl methylcellulose,
   f 2-15 wt. % of a polyacrylic acid polymer,
   g. 0.3-2 wt. % of magnesium stearate
Aspect 34. The gastro retentive solid oral dosage form of any one of Aspects 1-29, comprising:
   a. 4-7 wt. % of deutetrabenazine,
   b. 0.2-0.5 wt. % of a mixture of butylated hydroxyanisole and butylated hydroxytoluene,
   c. 20-50 wt. % of microcrystalline cellulose,
   d. 4-8 wt. % of copovidone,
   e. 20-40 wt. % of crospovidone,
   f. 5-20 wt. % of hydroxypropyl methylcellulose,
   g. 2-15 wt. % of a polyacrylic acid polymer,
   h. 0.3-2 wt. % of magnesium stearate
Aspect 35. The gastro retentive solid oral dosage form of any one of Aspects 1-29, comprising:
   a. 4-7 wt. % of deutetrabenazine,
   b. 0.2-0.5 wt. % of a mixture of butylated hydroxyanisole and butylated hydroxytoluene,
   c. 20-50 wt. % of microcrystalline cellulose,
   d. 4-8 wt. % of copovidone,
   e. 20-40 wt. % of crospovidone,
   f. 5-20 wt. % of hydroxypropyl methylcellulose,
   g. 2-15 wt. % of a polyacrylic acid polymer,
   h. 0.1-0.7 wt. % of sodium bicarbonate,
   i. 0.3-2 wt. % of magnesium stearate
Aspect 36. The gastro retentive solid oral dosage form of any one of the preceding Aspects, wherein the dosage form is a tablet.
Aspect 37. The gastro retentive solid oral dosage form of Aspect 36, wherein the tablet is a monolayered tablet.
Aspect 38. The gastro retentive solid oral dosage form of any one of the preceding Aspects, wherein the gastro retention in a subject is the result of one or more floatation mechanisms of gas generation and swelling when the dosage form is introduced to a gastric environment of the subject.
Aspect 39. The gastro retentive solid oral dosage form of Aspect 38, wherein the dosage form swells by about 200 wt. % of weight of the unswelled dosage form upon contact with gastric fluids.
Aspect 40. The gastro retentive solid oral dosage form of Aspects 38-39, wherein the dosage form floats in the gastric environment of the subject for up to 24 hours.
Aspect 41. The gastro retentive solid oral dosage form of any one of Aspects 38-40, wherein the dosage form floats in gastric environment of the subject for 6-20 hours.
Aspect 42. The gastro retentive solid oral dosage form of any one of the preceding Aspects, wherein not more than 40 wt. % of the deutetrabenazine is released after 2 hours when tested in 500 mL acid phosphate buffer at pH 3.0 using a USP II dissolution apparatus.
Aspect 43. The gastro retentive solid oral dosage form of Aspect 42, wherein not more than 20 wt. % of the deutetrabenazine is released after 2 hours when tested in 500 mL acid phosphate buffer at pH 3.0 using a USP II dissolution apparatus.
Aspect 44. The gastro retentive solid oral dosage form of any one of Aspects 1-41, wherein not more than 80 wt. % of the deutetrabenazine is released after 8 hours when tested in 500 mL acid phosphate buffer at pH 3.0 using a USP II dissolution apparatus.
Aspect 45. The gastro retentive solid oral dosage form of Aspect 44, wherein not more than 60 wt. % of the deutetrabenazine is released after 8 hours when tested in 500 mL acid phosphate buffer at pH 3.0 using a USP II dissolution apparatus.
Aspect 46. The gastro retentive solid oral dosage form of any one of the preceding Aspects, for the use in the treatment of a VMAT2 mediated disorder.
Aspect 47. A method of treating a VMAT2 mediated disorder in a subject in need thereof comprising orally administering, on a once daily basis to the subject, a gastro retentive solid oral dosage form according to any one of Aspects 1-45.
Aspect 48. The dosage form of Aspect 46 or the method of Aspect 47, wherein the VMAT2 mediated disorder is hyperkinetic movement disorder.
Aspect 49. The dosage form or the method of Aspect 48, wherein the hyperkinetic movement disorder is chronic hyperkinetic movement disorder.
Aspect 50. The dosage form or the method of Aspect 49, wherein the chronic hyperkinetic movement disorder is selected from Huntington's disease, Tardive dyskinesia, and dyskinesia in cerebral palsy.
Aspect 51. The dosage form or the method of any one of Aspects 46-50, wherein single dose administration of the once daily gastro retentive solid oral dosage form comprising a total amount of 6 mg of deutetrabenazine, provides an in vivo plasma profile for total α- and β-dihydrodeutetrabenazine that includes a geometric mean AUC_{0-inf} of about 91,250 to 142,750 h*pg/mL.
Aspect 52. The dosage form or the method of any one of Aspects 46-50, wherein single dose administration of the once daily gastro retentive solid oral dosage form comprising a total amount of 6 mg of deutetrabenazine, provides an in vivo plasma profile for total α- and β-dihydrodeutetrabenazine that includes a geometric mean Cₘₐₓ of less than about 4,600 pg/mL.
Aspect 53. The dosage form or the method of any one of Aspects 46-50, wherein single dose administration of the once daily gastro retentive solid oral dosage form comprising a total amount of 12 mg of deutetrabenazine, provides an in vivo plasma profile for total α- and β-dihydrodeutetrabenazine that includes a geometric mean AUC_{0-inf} of about 182,500 to 285,500 h*pg/mL.
Aspect 54. The dosage form or the method of any one of Aspects 46-50, wherein single dose administration of the once daily gastro retentive solid oral dosage form comprising a total amount of 12 mg of deutetrabenazine, provides an in vivo plasma profile for total α- and β-dihydrodeutetrabenazine that includes a geometric mean Cₘₐₓ of less than about 9,200 pg/mL.
Aspect 55. The dosage form or the method of any one of Aspects 46-50, wherein single dose administration of the once daily gastro retentive solid oral dosage form comprising a total amount of 24 mg of deutetrabenazine, provides an in vivo plasma profile for total α- and β-dihydrodeutetrabenazine that includes a geometric mean AUC_{0-inf} of about 365,000 to 571,000 h*pg/mL.
Aspect 56. The dosage form or the method of any one of Aspects 46-50, wherein single dose administration of the once daily gastro retentive solid oral dosage form comprising a total amount of 24 mg of deutetrabenazine, provides an in vivo plasma profile for total α- and β-dihydrodeutetrabenazine that includes a geometric mean Cₘₐₓ of less than about 18,400 pg/mL.
Aspect 57. The dosage form or the method of any one of Aspects 46-50, wherein single dose administration of the once daily gastro retentive solid oral dosage form comprising a total amount of 36 mg of deutetrabenazine, provides an in vivo plasma profile for total α- and β-dihydrodeutetrabenazine that includes a geometric mean AUC_{0-inf} of about 547,500 to 856,500 h*pg/mL.
Aspect 58. The dosage form or the method of any one of Aspects 46-50, wherein single dose administration of the once daily gastro retentive solid oral dosage form comprising a total amount of 36 mg of deutetrabenazine, provides an in vivo plasma profile for total α- and β-dihydrodeutetrabenazine that includes a geometric mean Cₘₐₓ of less than about 27,600 pg/mL.
Aspect 59. The dosage form or the method of any one of Aspects 46-50, wherein single dose administration of the once daily gastro retentive solid oral dosage form comprising a total amount of 48 mg of deutetrabenazine, provides an in vivo plasma profile for total α- and β-dihydrodeutetrabenazine that includes a geometric mean AUC_{0-inf} of about 730,000 to 1,142,000 h*pg/mL.
Aspect 60. The dosage form or the method of any one of Aspects 46-50, wherein single dose administration of the once daily gastro retentive solid oral dosage form comprising a total amount of 48 mg of deutetrabenazine, provides an in vivo plasma profile for total α- and β-dihydrodeutetrabenazine that includes a geometric mean Cₘₐₓ of less than about 36,800 pg/mL.
Aspect 61. The dosage form or the method of any one of Aspects 46-50, wherein administration of the once daily gastro retentive solid oral dosage form comprising a total amount of 6 mg of deutetrabenazine, provides an in vivo plasma profile for total α- and β-dihydrodeutetrabenazine at steady state that includes a mean AUC₀₋₂₄ of about 102,500 to 200,000 h*pg/mL.
Aspect 62. The dosage form or the method of any one of Aspects 46-50, wherein administration of the once daily gastro retentive solid oral dosage form comprising a total amount of 6 mg of deutetrabenazine, provides an in vivo plasma profile for total α- and β-dihydrodeutetrabenazine at steady state that includes a mean Cₘₐₓ of less than about 10,000 pg/mL.
Aspect 63. The dosage form or the method of any one of Aspects 46-50, wherein administration of the once daily gastro retentive solid oral dosage form comprising a total amount of 12 mg of deutetrabenazine, provides an in vivo plasma profile for total α- and β-dihydrodeutetrabenazine at steady state that includes a mean AUC₀₋₂₄ of about 205,000 to 400,000 h*pg/mL.
Aspect 64. The dosage form or the method of any one of Aspects 46-50, wherein administration of the once daily gastro retentive solid oral dosage form comprising a total amount of 12 mg of deutetrabenazine, provides an in vivo plasma profile for total α- and β-dihydrodeutetrabenazine at steady state that includes a mean Cₘₐₓ of less than about 20,000 pg/mL.
Aspect 65. The dosage form or the method of any one of Aspects 46-50, wherein administration of the once daily gastro retentive solid oral dosage form comprising a total amount of 24 mg of deutetrabenazine, provides an in vivo plasma profile for total α- and β-dihydrodeutetrabenazine at steady state that includes a mean AUC₀₋₂₄ of about 410,000 to 800,000 h*pg/mL.
Aspect 66. The dosage form or the method of any one of Aspects 46-50, wherein administration of the once daily gastro retentive solid oral dosage form comprising a total amount of 24 mg of deutetrabenazine, provides an in vivo plasma profile for total α- and β-dihydrodeutetrabenazine at steady state that includes a mean Cₘₐₓ of less than about 40,000 pg/mL.
Aspect 67. The dosage form or the method of any one of Aspects 46-50, wherein administration of the once daily gastro retentive solid oral dosage form comprising a total amount of 36 mg of deutetrabenazine, provides an in vivo plasma profile for total α- and β-dihydrodeutetrabenazine at steady state that includes a mean AUC₀₋₂₄ of about 615,000 to 1.200,000 h*pg/mL.
Aspect 68. The dosage form or the method of any one of Aspects 46-50, wherein administration of the once daily gastro retentive solid oral dosage form comprising a total amount of 36 mg of deutetrabenazine, provides an in vivo plasma profile for total α- and β-dihydrodeutetrabenazine at steady state that includes a mean Cₘx of less than about 60,000 pg/mL.
Aspect 69. The dosage form or the method of any one of Aspects 46-50, wherein administration of the once daily gastro retentive solid oral dosage form comprising a total amount of 48 mg of deutetrabenazine, provides an in vivo plasma profile for total α- and β-dihydrodeutetrabenazine at steady state that includes a mean AUC₀₋₂₄ of about 820,000 to 1,600,000 h*pg/mL.
Aspect 70. The dosage form or the method of any one of Aspects 46-50, wherein administration of the once daily gastro retentive solid oral dosage form comprising a total amount of 48 mg of deutetrabenazine, provides an in vivo plasma profile for total α- and β-dihydrodeutetrabenazine at steady state that includes a mean Cₘₐₓ of less than about 80,000 pg/mL.
Aspect 71. The dosage form or the method of any one of Aspects 46-50, wherein following the administration of the gastro retentive solid oral dosage form according to any one of Aspects 1-45, not more than 40 wt. % of the deutetrabenazine is released after 2 hours when tested in 500 mL acid phosphate buffer at pH 3.0 using a USP II dissolution apparatus.
Aspect 72. The dosage form or the method of any one of Aspects 46-50, wherein following the administration of a gastro retentive solid oral dosage form according to any one of Aspects 1-45, not more than 20 wt. % of the deutetrabenazine is released after 2 hours when tested in 500 mL acid phosphate buffer at pH 3.0 using a USP II dissolution apparatus.
Aspect 73. The dosage form or the method of any one of Aspects 46-50, wherein following the administration of a gastro retentive solid oral dosage form according to any one of Aspects 1-45, not more than 80 wt. % of the deutetrabenazine is released after 8 hours when tested in 500 mL acid phosphate buffer at pH 3.0 using a USP II dissolution apparatus.
Aspect 74. The dosage form or the method of any one of Aspects 46-50, wherein following the administration of a gastro retentive solid oral dosage form according to any one of Aspects 1-45, not more than 60 wt. % of the deutetrabenazine is released after 8 hours when tested in 500 mL acid phosphate buffer at pH 3.0 using a USP II dissolution apparatus.
Aspect 75. The dosage form or the method of any one of Aspects 46-74, wherein the dosage form is administered with food.
Aspect 76. The dosage form or the method of any one of Aspects 46-74, wherein the dosage form is administered independent of food intake.

1. A controlled release gastro retentive solid oral dosage form for once daily administration of deutetrabenazine comprising:
a. an amount of deutetrabenazine;
b. at least two control release polymers, each independently having a viscosity of 2,000 cPs or more; and
c. a pharmaceutically acceptable excipient comprising at least one disintegrant;
wherein the total amount of control release polymers is at least 30 wt. % relative to the total weight of the dosage form.

2. (canceled)

3. (canceled)

4. (canceled)

5. The gastro retentive solid oral dosage form of claim 1, further comprising at least one additional control release polymer, each independently having a viscosity of about 3 to about 80,000 cPs.

6. The gastro retentive solid oral dosage form of claim 1, wherein the control release polymer or an additional control release polymer comprises a water soluble polymer, a water insoluble polymer or mixtures thereof.

7. (canceled)

8. (canceled)

9. (canceled)

10. (canceled)

11. (canceled)

12. (canceled)

13. The gastro retentive solid oral dosage form of claim 1, comprising a control release polymer mixture of copovidone, hydroxypropyl methylcellulose and a polyacrylic acid polymer.

14. The gastro retentive solid oral dosage form of claim 1, wherein the at least one disintegrant comprises: croscarmellose sodium, alginic acid, microcrystalline cellulose, crospovidone, polacrilin potassium, sodium starch glycolate, low-substituted hydroxypropyl cellulose, starch, or mixtures thereof.

15. (canceled)

16. (canceled)

17. The gastro retentive solid dosage form of claim 1, wherein the pharmaceutically acceptable excipient further comprises a diluent, a binder, a gas-generating agent, an antioxidant, a lubricant or combinations thereof.

18. (canceled)

19. (canceled)

20. (canceled)

21. (canceled)

22. (canceled)

23. The gastro retentive solid oral dosage form of claim 1, wherein the pharmaceutically acceptable excipient comprises a binder, and wherein the binder comprises, microcrystalline cellulose, starch, gelatin, agar, natural and synthetic gums or any mixture thereof.

24. (canceled)

25. The gastro retentive solid oral dosage form claim 1, comprising 2-15 wt. %, 3-10 wt % or 4-7 wt. % deutetrabenazine, relative to the total weight of the dosage form.

26. The gastro retentive solid oral dosage form of claim 1, wherein the total amount of deutetrabenazine in the dosage form is from about 6 mg to about 48 mg.

27. (canceled)

28. (canceled)

29. (canceled)

30. (canceled)

31. (canceled)

32. The gastro retentive solid oral dosage form of claim 1, wherein the deutetrabenazine is micronized deutetrabenazine having a mean particle size of about 1 m to about 30 m in diameter.

33. (canceled)

34. (canceled)

35. (canceled)

36. The gastro retentive solid oral dosage form of claim 1, comprising:
a. 4-7 wt. % of deutetrabenazine relative to the total weight of the dosage form,
b. 20-40 wt. % of crospovidone relative to the total weight of the dosage form,
c. 20-50 wt. % of hydroxypropyl methylcellulose relative to the total weight of the dosage form,
d. 15-45 wt. % of a polyacrylic acid polymer relative to the total weight of the dosage form,
e. 0.3-2 wt. % of magnesium stearate relative to the total weight of the dosage form.

37. (canceled)

38. The gastro retentive solid oral dosage form of claim 1, comprising:
a. 4-7 wt. % of deutetrabenazine relative to the total weight of the dosage form,
b. 5-20 wt. % of microcrystalline cellulose relative to the total weight of the dosage form
c. 10-30 wt. % of copovidone relative to the total weight of the dosage form,
d. 20-40 wt. % of crospovidone relative to the total weight of the dosage form,
e. 20-50 wt. % of hydroxypropyl methylcellulose relative to the total weight of the dosage form,
f. 2-15 wt. % of a polyacrylic acid polymer relative to the total weight of the dosage form,
g. 0.3-2 wt. % of magnesium stearate relative to the total weight of the dosage form.

39. The gastro retentive solid oral dosage form of claim 1, comprising:
a. 4-7 wt. % of deutetrabenazine relative to the total weight of the dosage form,
b. 0.2-0.5 wt. % of a mixture of butylated hydroxyanisole and butylated hydroxytoluene relative to the total weight of the dosage form,
c. 20-50 wt. % of microcrystalline cellulose relative to the total weight of the dosage form,
d. 4-8 wt. % of copovidone relative to the total weight of the dosage form,
e. 20-40 wt. % of crospovidone relative to the total weight of the dosage form,
f. 5-20 wt. % of hydroxypropyl methylcellulose relative to the total weight of the dosage form,
g. 2-15 wt. % of a polyacrylic acid polymer relative to the total weight of the dosage form,
h. 0.3-2 wt. % of magnesium stearate relative to the total weight of the dosage form.

40. (canceled)

41. The gastro retentive solid oral dosage form of claim 1, wherein the dosage form is a tablet.

42. (canceled)

43. The gastro retentive solid oral dosage form of claim claim 1, wherein the gastro retention in a subject is the result of one or more floatation mechanisms when the dosage form is introduced to a gastric environment of the subject.

44. (canceled)

45. (canceled)

46. (canceled)

47. The gastro retentive solid oral dosage form of claim 1, wherein not more than 40 wt. % of the deutetrabenazine is released after 2 hours when tested in 500 mL acid phosphate buffer at pH 3.0 using a USP II dissolution apparatus, not more than 80 wt. % of the deutetrabenazine is released after 8 hours when tested in 500 mL acid phosphate buffer at pH 3.0 using a USP II dissolution apparatus, or both.

48. (canceled)

49. (canceled)

50. (canceled)

51. (canceled)

52. A method of treating a VMAT2 mediated disorder in a subject in need thereof comprising orally administering, on a once daily basis to the subject, a gastro retentive solid oral dosage form according to claim 1.

53. The method of claim 52, wherein the VMAT2 mediated disorder is hyperkinetic movement disorder.

54. (canceled)

55. (canceled)

56. The gastro retentive solid oral dosage form of claim 1,
wherein single dose administration of the once daily gastro retentive solid oral dosage form comprising a total amount of 6 mg of deutetrabenazine, provides an in vivo plasma profile for total α- and β-dihydrodeutetrabenazine that includes a geometric mean AUC_{0-inf} of about 91,250 to 142,750 h*pg/mL, or
wherein single dose administration of the once daily gastro retentive solid oral dosage form comprising a total amount of 6 mg of deutetrabenazine, provides an in vivo plasma profile for total α- and β-dihydrodeutetrabenazine that includes a geometric mean Cₘₐₓ of less than about 4,600 pg/mL, or
wherein administration of the once daily gastro retentive solid oral dosage form comprising a total amount of 6 mg of deutetrabenazine, provides an in vivo plasma profile for total α- and β-dihydrodeutetrabenazine at steady state that includes a mean AUC₀₋₂₄ of about 102,500 to 200,000 h*pg/mL, or
wherein administration of the once daily gastro retentive solid oral dosage form comprising a total amount of 6 mg of deutetrabenazine, provides an in vivo plasma profile for total α- and β-dihydrodeutetrabenazine at steady state that includes a mean Cₘₐₓ of less than about 10,000 pg/mL.

57. (canceled)

58. The gastro retentive solid oral dosage form of claim 1, wherein single dose administration of the once daily gastro retentive solid oral dosage form comprising a total amount of 12 mg of deutetrabenazine, provides an in vivo plasma profile for total α- and β-dihydrodeutetrabenazine that includes a geometric mean AUC_{0-inf} of about 182,500 to 285,500 h*pg/mL, or
wherein single dose administration of the once daily gastro retentive solid oral dosage form comprising a total amount of 12 mg of deutetrabenazine, provides an in vivo plasma profile for total α- and β-dihydrodeutetrabenazine that includes a geometric mean Cₘₐₓ of less than about 9,200 pg/mL, or
wherein administration of the once daily gastro retentive solid oral dosage form comprising a total amount of 12 mg of deutetrabenazine, provides an in vivo plasma profile for total α- and β-dihydrodeutetrabenazine at steady state that includes a mean AUC₀₋₂₄ of about 205,000 to 400,000 h*pg/mL, or
wherein administration of the once daily gastro retentive solid oral dosage form comprising a total amount of 12 mg of deutetrabenazine, provides an in vivo plasma profile for total α- and β-dihydrodeutetrabenazine at steady state that includes a mean Cₘₐₓ of less than about 20,000 pg/mL.

59. (canceled)

60. The gastro retentive solid oral dosage form of claim 1, wherein single dose administration of the once daily gastro retentive solid oral dosage form comprising a total amount of 24 mg of deutetrabenazine, provides an in vivo plasma profile for total α- and β-dihydrodeutetrabenazine that includes a geometric mean AUC_{0-inf} of about 365,000 to 571,000 h*pg/mL, or
wherein single dose administration of the once daily gastro retentive solid oral dosage form comprising a total amount of 24 mg of deutetrabenazine, provides an in vivo plasma profile for total α- and β-dihydrodeutetrabenazine that includes a geometric mean Cₘₐₓ of less than about 18,400 pg/mL, or
wherein administration of the once daily gastro retentive solid oral dosage form comprising a total amount of 24 mg of deutetrabenazine, provides an in vivo plasma profile for total α- and β-dihydrodeutetrabenazine at steady state that includes a mean AUC₀₋₂₄ of about 410,000 to 800,000 h*pg/mL, or
wherein administration of the once daily gastro retentive solid oral dosage form comprising a total amount of 24 mg of deutetrabenazine, provides an in vivo plasma profile for total α- and β-dihydrodeutetrabenazine at steady state that includes a mean Cₘₐₓ of less than about 40,000 pg/mL.

61. (canceled)

62. The gastro retentive solid oral dosage form of claim 1, wherein single dose administration of the once daily gastro retentive solid oral dosage form comprising a total amount of 36 mg of deutetrabenazine, provides an in vivo plasma profile for total α- and β-dihydrodeutetrabenazine that includes a geometric mean AUC_{0-inf} of about 547,500 to 856,500 h*pg/mL, or
wherein single dose administration of the once daily gastro retentive solid oral dosage form comprising a total amount of 36 mg of deutetrabenazine, provides an in vivo plasma profile for total α- and β-dihydrodeutetrabenazine that includes a geometric mean Cₘₐₓ of less than about 27,600 pg/mL, or
wherein administration of the once daily gastro retentive solid oral dosage form comprising a total amount of 36 mg of deutetrabenazine, provides an in vivo plasma profile for total α- and β-dihydrodeutetrabenazine at steady state that includes a mean AUC₀₋₂₄ of about 615,000 to 1,200,000 h*pg/mL, or
wherein administration of the once daily gastro retentive solid oral dosage form comprising a total amount of 36 mg of deutetrabenazine, provides an in vivo plasma profile for total α- and β-dihydrodeutetrabenazine at steady state that includes a mean Cₘₐₓ of less than about 60,000 pg/mL.

63. (canceled)

64. The gastro retentive solid oral dosage form of claim 1, wherein single dose administration of the once daily gastro retentive solid oral dosage form comprising a total amount of 48 mg of deutetrabenazine, provides an in vivo plasma profile for total α- and β-dihydrodeutetrabenazine that includes a geometric mean AUC_{0-inf} of about 730,000 to 1,142,000 h*pg/mL, or
wherein single dose administration of the once daily gastro retentive solid oral dosage form comprising a total amount of 48 mg of deutetrabenazine, provides an in vivo plasma profile for total α- and β-dihydrodeutetrabenazine that includes a geometric mean Cₘₐₓ of less than about 36,800 pg/mL, or
wherein administration of the once daily gastro retentive solid oral dosage form comprising a total amount of 48 mg of deutetrabenazine, provides an in vivo plasma profile for total α- and β-dihydrodeutetrabenazine at steady state that includes a mean AUC₀₋₂₄ of about 820,000 to 1,600,000 h*pg/mL, or
wherein administration of the once daily gastro retentive solid oral dosage form comprising a total amount of 48 mg of deutetrabenazine, provides an in vivo plasma profile for total α- and β-dihydrodeutetrabenazine at steady state that includes a mean Cₘₐₓ of less than about 80,000 pg/mL.

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. The gastro retentive solid oral dosage form according to claim 1, wherein following the administration of the gastro retentive solid oral dosage form, about 50 wt. % of the deutetrabenazine is released after 7 hours when tested in vitro in 500 mL acid phosphate buffer at pH 3.0 using a USP II dissolution apparatus.

77. (canceled)

78. (canceled)
